(19) United States
(12) Patent Application Publication
(10) Pub. No.: US 2008/0020981 A1
(43) Pub. Date: Jan. 24, 2008

(54) Non-nucleoside reverse transcriptase inhibitors

(75) Inventor: Zachary Kevin Sweeney, Redwood City, CA (US)

Correspondence Address: ROCHE PALO ALTO LLC;PATENT LAW DEPT. M/S A2-250, 3431 HILLVIEW AVENUE, PALO ALTO, CA, 94304, US

(73) Assignee: Roche Palo Alto LLC

(21) Appl. No.: 11/880,229

(22) Filed: Jul. 20, 2007

Related U.S. Application Data
(60) Provisional application No. 60/832,482, filed on Jul. 21, 2006.

(51) Int. Cl. A61K 31/075; A61K 31/165; A61K 31/34; A61K 31/415; A61K 31/425; A61K 31/44; A61K 31/445; A61K 31/50; A61K 31/505; A61K 31/535; A61K 38/00; A61P 43/00; C07C 229/00; C07C 233/00; C07D 233/38

(52) U.S. Cl. 514/12; 514/230.5; 514/248; 514/27; 514/274; 514/293; 514/318; 514/365; 514/391; 514/471; 514/622; 514/716; 548/316.7; 560/42; 564/186

(57) ABSTRACT

The present invention provides for compounds useful for treating an HIV infection, or preventing an HIV infection, or treating AIDS or ARC. The compounds of the invention are of formula I wherein R¹, R², R³, R⁴, X¹ and X² are as herein defined. Also disclosed in the present invention are methods of treating an HIV infection with compounds defined herein and pharmaceutical compositions containing said compounds.

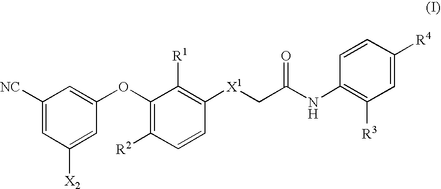

### CROSS REFERENCE TO RELATED APPLICATIONS

This application is claims benefit of U.S. Provisional Application No. 60/832,482, filed Jul. 21, 2006, which is hereby incorporated by reference in its entirety.

### FIELD OF THE INVENTION

The invention relates to the field of antiviral therapy and, in particular, to non-nucleoside compounds that inhibit HIV reverse transcriptase and are useful for treating Human Immunodeficiency Virus (HIV) mediated diseases. The invention provides novel N-phenyl phenoxyacetamide compounds according to formula I, for treatment or prophylaxis of HIV mediated diseases, AIDS or ARC, employing said compounds in monotherapy or in combination therapy.

### BACKGROUND OF THE INVENTION

The human immunodeficiency virus HIV is the causative agent of acquired immunodeficiency syndrome (AIDS), a disease characterized by the destruction of the immune system, particularly of the CD4⁺ T-cell, with attendant susceptibility to opportunistic infections. HIV infection is also associated with a precursor AIDS-related complex (ARC), a syndrome characterized by symptoms such as persistent generalized lymphadenopathy, fever and weight loss.

In common with other retroviruses, the HIV genome encodes protein precursors known as gag and gag-pol which are processed by the viral protease to afford the protease, reverse transcriptase (RT), endonuclease/integrase and mature structural proteins of the virus core. Interruption of this processing prevents the production of normally infectious virus. Considerable efforts have been directed towards the control of HIV by inhibition of virally encoded enzymes.

Two enzyme have extensively studied for HIV-1 chemotherapy: HIV protease and HIV reverse transcriptase. (J. S. G. Montaner et al., Antiretroviral therapy: ‘the state of the art’ Biomed & Pharmacother. 1999 53:63-72; R. W. Shafer and D. A. Vuitton, Highly active retroviral therapy (HAART)for the treatment of infection with human immunodeficiency virus type, Biomed. & Pharmacother. 1999 53:73-86; E. De Clercq, New Developments in Anti-HIV Chemotherap. Curr. Med. Chem. 2001 8:1543-1572). Two general classes of RTI inhibitors have been identified: nucleoside reverse transcriptase inhibitors (NRTI) and non-nucleoside reverse transcriptase inhibitors. Currently the CCR5 co-receptor has emerged as a potential target for anti-HIV chemotherapy (D. Chantry, Expert Opin. Emerg. Drugs 2004 9(1):1-7; C. G. Barber, Curr. Opin. Invest. Drugs 2004 5(8):851-861; D. Schols, Curr. Topics Med. Chem. 2004 4(9):883-893; N. A. Meanwell and J. F. Kadow, Curr. Opin. Drug Discov. Dev. 2003 6(4):451-461). N-substituted hydroxy pyrimidinone carboxamide inhibitors of HIV-1 integrase inhibitors have been disclosed by B. Crescenzi et al. in WO2003/035077, published May 1, 2003, and MK-0518 is nearing approval

NRTIs typically are 2′,3′-dideoxynucleoside (ddN) analogs which must be phosphorylated prior to interacting with viral RT. The corresponding triphosphates function as competitive inhibitors or alternative substrates for viral RT. After incorporation into nucleic acids the nucleoside analogs terminate the chain elongation process. HIV reverse transcriptase has DNA editing capabilities which enable resistant strains to overcome the blockade by cleaving the nucleoside analog and continuing the elongation. Currently clinically used NRTIs include zidovudine (AZT), didanosine (ddI), zalcitabine (ddC), stavudine (d4T), lamivudine (3TC) and tenofovir (PMPA). Two enzyme have extensively studied for HIV-1 chemotherapy: HIV protease and HIV reverse transcriptase. (J. S. G. Montaner et al., Antiretroviral therapy: ‘the state of the art’ Biomed & Pharmacother. 1999 53:63-72; R. W. Shafer and D. A. Vuitton, Highly active retroviral therapy (HAART) for the treatment of infection with human immunodeficiency virus type, Biomed. & Pharmacother. 1999 53:73-86; E. De Clercq, New Developments in Anti-HIV Chemotherap. Curr. Med. Chem. 2001 8:1543-1572). Two general classes of RTI inhibitors have been identified: nucleoside reverse transcriptase inhibitors (NRTI) and non-nucleoside reverse transcriptase inhibitors. Currently the CCR5 co-receptor has emerged as a potential target for anti-HIV chemotherapy (D. Chantry, Expert Opin. Emerg. Drugs 2004 9(1):I-7; C. G. Barber, Curr. Opin. Invest. Drugs 2004 5(8):851-861; D. Schols, Curr. Topics Med. Chem. 2004 4(9):883-893; N. A. Meanwell and J. F. Kadow, Curr. Opin. Drug Discov. Dev. 2003 6(4):451-461). N-substituted hydroxy pyrimidinone carboxamide inhibitors of HIV-1 integrase inhibitors have been disclosed by B. Crescenzi et al. in WO2003/035077, published May 1, 2003, and MK-0518 is nearing approval

NNRTIs were first discovered in 1989. NNRTI are allosteric inhibitors which bind reversibly at a nonsubstrate-binding site on the HIV reverse transcriptase thereby altering the shape of the active site or blocking polymerase activity (R. W. Buckheit, Jr., Non-nucleoside reverse transcriptase inhibitors. perspectives for novel therapeutic compounds and strategies for treatment of HIV infection, Expert Opin. Investig. Drugs 2001 10(8)1423-1442; E. De Clercq, The role of non-nucleoside reverse transcriptase inhibitors (NNRTIs) in the therapy of HIV infection, Antiviral Res. 1998 38:153-179; E. De Clercq, New Developments in Anti-HIV Chemotherapy, Current medicinal Chem. 2001 8(13):1543-1572; G. Moyle, The Emerging Roles of Non-Nucleoside Reverse Transcriptase Inhibitors in Antiviral Therapy, Drugs 2001 61 (1): 19-26). Although over thirty structural classes of NNRTIs have been identified in the laboratory, only three compounds have been approved for HIV therapy: efavirenz, nevirapine and delavirdine. Two enzyme have extensively studied for HIV-1 chemotherapy: HIV protease and HIV reverse transcriptase. (J. S. G. Montaner et al., Antiretroviral therapy: ‘the state of the art’, Biomed & Pharmacother. 1999 53:63-72; R. W. Shafer and D. A. Vuitton, Highly active retroviral therapy (HAART)for the treatment of infection with human immunodeficiency virus type, Biomed. & Pharmacother. 1999 53:73-86; E. De Clercq, New Developments in Anti-HIV Chemotherap. Curr. Med. Chem. 2001 8:1543-1572). Two general classes of RTI inhibitors have been identified: nucleoside reverse transcriptase inhibitors (NRTI) and non-nucleoside reverse transcriptase inhibitors. Currently the CCR5 co-receptor has emerged as a potential target for anti-HIV chemotherapy (D. Chantry, Expert Opin. Emerg. Drugs 2004 9(1):1-7; C. G. Barber, Curr. Opin. Invest. Drugs 2004 5(8):851-861; D. Schols, Curr. Topics Med. Chem. 2004 4(9):883-893; N. A. Meanwell and J. F. Kadow, Curr. Opin. Drug Discov. Dev. 2003 6(4):451-461). N-substituted hydroxy pyrimidinone carboxamide inhibitors of HIV-1 integrase inhibitors have been disclosed by B. Crescenzi et al. in WO2003/035077, published May 1, 2003, and MK-0518 is nearing approval

Initially viewed as a promising class of compounds, in vitro and in vivo studies quickly revealed the NNRTIs presented a low barrier to the emergence of drug resistant HIV strains and class-specific toxicity. Drug resistance frequently develops with only a single point mutation in the RT. While combination therapy with NRTIs, PIs and NNRTIs has, in many cases, dramatically lowered viral loads and slowed disease progression, significant therapeutic problems remain. (R. M. Gulick, Eur. Soc. Clin. Microbiol. and Inf. Dis. 2003 9(3): 186-193) The cocktails are not effective in all patients, potentially severe adverse reactions often occur and the rapidly reproducing HIV virus has proven adroit at creating mutant drug-resistant variants of wild type protease and reverse transcriptase. There remains a need for safer drugs with activity against wild type and commonly occurring resistant strains of HIV.

Certain N-phenyl phenyloxyacetamide compounds have now been found to have a variety of desirable pharmacological properties.

In WO2002070471 published Sep. 12, 2002, B. Cezanne et al. disclose phenyl derivatives that are inhibitors of coagulation Factor Xa and useful for prophylaxis and/or therapy of thromboembolytic disorders or for the treatment of tumors.

In U.S. Pat. No. 6,531,291 C. Kabbash et al. disclose methods of selecting compounds which inhibit the enzymatic activity of enoyl reductase.

In BE 854-683 (Derwent 82937Y/47) K. G. Merckle disclose (N)-acyl-metoclopramide derivatives useful as e.g. anti-inflammatories, anti-allergic agents and muscle relaxants.

In WO9316036 published Sep. 12, 2002, R. Anderskewitz et al. disclose new amidine derivatives that are LTB antagonists and useful for treating allergic disorders.

In EP0791576 published Aug. 27, 1997, T. S. Abram et al. disclose new benzoic acid compounds that are leukotriene antagonists and useful for the treatment of respiratory diseases.

In WO 2003075907 published Sep. 10, 2003, M.-P. DeBethune et al. disclose new N-substituted aniline derivatives useful as viral entry inhibitors to treat, e.g., human immunodeficiency virus infections or acquired immunodeficiency syndrome.

In U.S. Pat. No. 5,741,926 published Apr. 21, 1998, D. E. Bierer and A. G. Dubenko disclose aniline erivatives which exhibit antihyperglycemic activity.

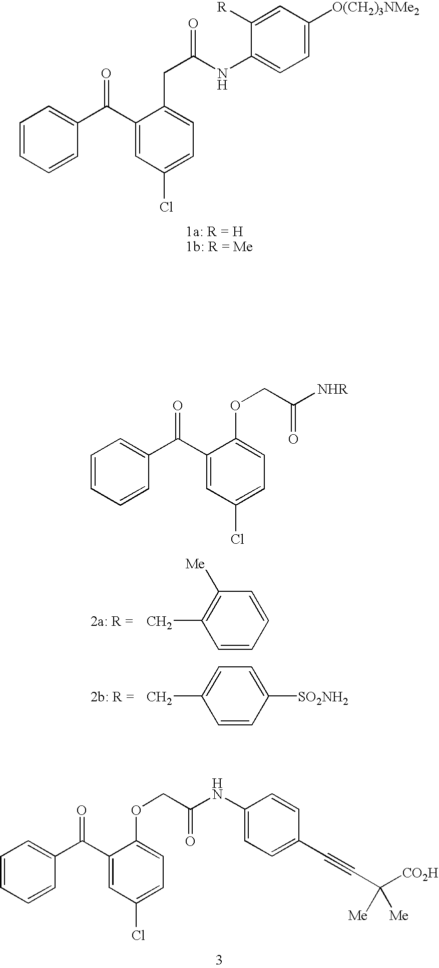

2-Benzoyl phenyl-N-[phenyl]-acetamide compounds 1a and 1b have been shown to inhibit HIV-1 reverse transcriptase (P. G. Wyatt et al., J. Med. Chem. 1995 38(10):1657-1665). Further screening identified related compounds, e.g. 2-benzoyl phenyloxy-N-[phenyl]-acetamide, 2a, and a sulfonamide derivative 2b which also inhibited reverse transcriptase (J. H. Chan et al., J. Med. Chem. 2004 47(5):1175-1182; ## et al., J. Med. Chem. 2006 K. R. Romines et al., J. Med. Chem. 2006 49(2): 727-739; C. L. Webster et al., WO01/17982). P. Bonneau et al. in US 20060069261 published Mar. 30, 2006 disclose 4-{4-[2-(2-benzoyl-phenoxy)-acetylamino]-phenyl}-2,2-dimethyl-but-3-ynoic acid compounds 3 which are inhibitors of HIV reverse transcriptase.

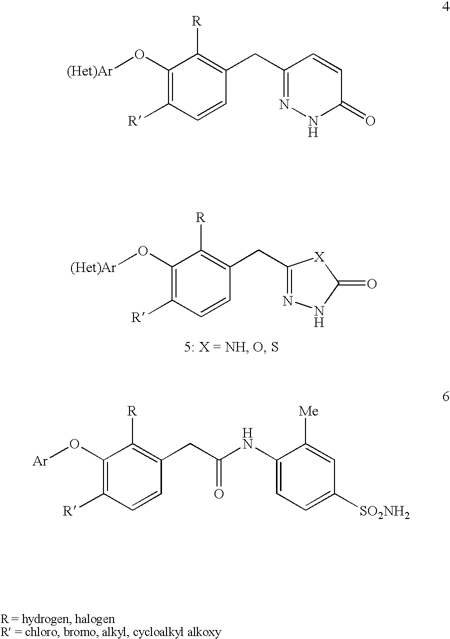

Pyridazinone non-nucleoside reverse transcriptase inhibitors 4 have been described by J. P. Dunn et al. in U.S. Pat. No. 7,18,718 issued Mar. 13, 2007 and by J. P. Dunn et al. in U.S. Publication No. 2005021554 filed Mar. 22, 2005. 5-Aralkyl-2,4-dihydro-[1,2,4]triazol-3-one, 5-aralkyl-3H-[1,3,4]oxadiazol-2-one and 5-aralkyl-3H-[1,3,4]thiadiazol-2-one non-nucleoside reverse transcriptase inhibitors 5 have been disclosed by J. P. Dunn et al. in U.S. Pat. No. 7,208,509 issued Apr. 24, 2007 and by J. P. Dunn et al. in U.S. Publication No. 20060025462 filed Jun. 27, 2005. Related compounds are disclosed by Y. D. Saito et al. in U.S. Ser. No. 60/722,335. Phenylacetamide non-nucleoside reverse transcriptase inhibitors 6 have been disclosed by J. P. Dunn et al. in U.S. Pat. No. 7,166,738 issued Jan. 27, 2007 and methods for treating retroviral infection with phenylacetamide compounds have been disclosed by J. P. Dunn et al. in U.S. Publication No. 20050239880 published Oct. 27, 2005; T. Mirzadegan and T. Silva in U.S. Publication No. 20070088053 published Apr. 19, 2007; and Z. K. Sweeney and T. Silva in U.S. Publication No. 20070088015 published Apr. 19, 2007. These applications are hereby incorporated by reference in their entirety.

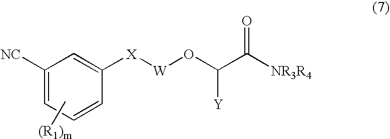

In WO2006/067587 published Jun. 26, 2006, L. H. Jones et al. disclose biaryl ether derivatives 7 and compositions containing them which bind to the enzyme reverse transcriptase and are modulators, especially inhibitors, thereof. In U.S. Patent Publication 2007/0021442 published Jan. 25, 2007, S. A. Saggar et al. disclose diphenyl ether HIV-1 reverse transcriptase inhibitors.

### SUMMARY OF THE INVENTION

The present invention relates to formula I wherein:

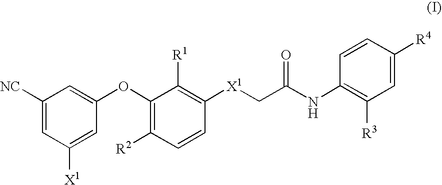

R¹ is fluorine or hydrogen;

R² is hydrogen, chloro, bromo, C₁₋₃ alkyl, C₃₋₅ cycloalkyl or C₁₋₃ alkoxy;

X¹ is O or S;

X² is chloro, bromo, cyano, C₁₋₃ alkoxy, C₃₋₅ cycloalkyl or C₁₋₃ haloalkyl;

R³ is selected from the group consisting of C₁₋₆ alkyl, C₁₋₃ haloalkyl, C₁₋₆ alkoxy, C₁₋₆ haloalkoxy, C₃₋₅ cycloalkyl, halogen and cyano;

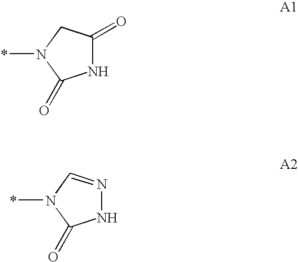

R⁴ is SONHR^{5a}R^{6a}, COX⁴, —C≡CC(Me)₂R⁸, A1 or A2;

X⁴ is OH or NR^{5b}R^{6b};

R^{5a} and R^{6a}
(i) taken independently, one of R^{5a} and R^{6a} is hydrogen or C₁₋₆ alkyl and the other of R^{5a} and R^{6a} is selected from the group consisting of hydrogen, C₁₋₆ alkyl and —C(═O)R⁷; or
(ii) taken together with the nitrogen atom to which they are attached form an azetidine, pyrrolidine, piperidine or azepine ring said azetidine, pyrrolidine, piperidine or azepine ring optionally substituted with hydroxy, amino, C₁₋₃ alkylamine or C₁₋₃ dialkylamine; or,
(iii) taken together are (CH₂)₂—X³—(CH₂)₂;

R^{5b} and R^{6b}
(i) are independently selected from the group consisting of hydrogen, C₁₋₆ alkyl, C₁₋₆ hydroxyalkyl, C₁₋₆ carboxyalkyl, (CH₂)ᵣNR^{5c}R^{6c} wherein r is 2 to 6, and SO₂—C₁₋₆ alkyl;
(ii) taken together with the nitrogen atom to which they are attached form an azetidine, pyrrolidine, piperidine or azepine ring said azetidine, pyrrolidine, piperidine or azepine ring optionally substituted hydroxy, amino, C₁₋₃ alkylamine or C₁₋₃ dialkylamine; or,
(iii) taken together are (CH₂)₂X³(CH₂)₂;

R^{5c} and R^{6c}
(i) are independently from the group consisting of hydrogen and C₁₋₆ alkyl,
(ii) together with the nitrogen atom to which they are attached form an azetidine, pyrrolidine, piperidine or azepine ring said azetidine, pyrrolidine, piperidine or azepine ring optionally substituted hydroxy, amino, C₁₋₃ alkylamine or C₁₋₃ dialkylamine, or
(iii) taken together are (CH₂)₂X³(CH₂)₂;

R^{5d} and R^{6d} are independently in each occurrence hydrogen, C₁₋₆ alkyl, C₁₋₆ hydroxyalkyl or C₁₋₆ carboxyalkyl;

X³ is O, S(O)ₚ or NR¹⁰;

R⁷ is hydrogen or C₁₋₆ alkyl;

R⁸ is OH, NR^{5d}R^{6d}, CO₂H, CONR^{5d}R^{5d} or C(═O)NR^{9a}C(═NR^{9b})NR^{9c}R^{9d};

R^{9a}, R^{9b}, R^{9c} and R^{9d} are (i) independently hydrogen or C₁₋₆ alkyl or (ii) R^{9a} and R^{9d} are independently hydrogen or C₁₋₆ alkyl and R^{9b} and R^{9c} together are C₂₋₄ alkylene;

R¹⁰ is hydrogen, C₁₋₆ alkyl or C₁₋₆ acyl;

p is 0 to 2; or,

a pharmaceutically acceptable salt thereof.

Compounds of formula I inhibit HIV reverse transcriptase and afford a method for prevention and treatment of HIV infections and the treatment of AIDS and/or ARC. HIV undergoes facile mutations of its genetic code resulting in strains with reduced susceptibility to therapy with current therapeutic options. The present invention also relates to compositions containing compounds of formula I useful for the prevention and treatment of HIV infections and the treatment of AIDS and/or ARC. The present invention further relates to compounds of formula I which are useful in mono therapy or combination therapy with other anti-viral agents.

### DETAILED DESCRIPTION OF THE INVENTION

In one embodiment of the present invention there is provided a compound according to formula I wherein R¹, R², R³, R⁴, R^{5a}, R^{5b}, R^{5c}, R^{5d}, R^{6a}, R^{6b}, R^{6c}, R^{6d}, R⁷, R⁸, R^{9a}, R^{9b}, R^{9c}, R^{9d}, R¹⁰, X¹, X², X³, X⁴, r and p are as defined herein above. The phrase “as defined herein above” refers to the broadest definition for each group as provided in the Summary of the Invention. In other embodiments provided below, substituents present in each embodiment which are not explicitly defined retain the broadest definition provided in the Summary of the Invention.

In a second embodiment of the present invention there is provided a compound according to formula I wherein R⁴ is SO₂NHR^{5a}R^{6a} or COX⁴.

In a third embodiment of the present invention there is provided a compound according to formula I wherein X¹ is O, R¹ is fluoro, R³ is chloro, bromo, or methyl and R⁴ is SO₂NHR^{5a}R^{6a}.

In a fourth embodiment of present invention there is provided a compound according to formula I wherein X¹ is O, X² is chloro, bromo, difluoromethyl or cyano, R¹ is fluoro, R² is methyl, ethyl, methoxy, chloro or bromo, R³ is chloro, bromo, or methyl and R⁴ is SO₂NHR^{5a}R^{6a}.

In a fifth embodiment of the present invention there is provided a compound according to formula I wherein X¹ is O, X² is chloro, bromo, difluoromethyl or cyano, R¹ is fluoro, R² is methyl, ethyl, methoxy, chloro or bromo, R³ is chloro, bromo, or methyl, R⁴ is SO₂NHR^{5a}R^{6a}, R^{5a} is hydrogen, R^{6a} is hydrogen or R⁷C(═O) and R⁷ is C₁₋₁₀ alkyl.

In a sixth another embodiment of the present invention there is provided a compound according to formula I wherein X¹ is O, R¹ is fluoro, R³ is chloro, bromo or methyl and R⁴ is CONR^{5b}R^{6b}.

In a seventh another embodiment of the present invention there is provided a compound according to formula I wherein X¹ is O, X² is chloro, bromo, difluoromethyl or cyano, R¹ is fluoro, R² is methyl, ethyl, methoxy, chloro or bromo, R³ is chloro, bromo or methyl and R⁴ is CONR^{5b}R^{6b}.

In an eighth embodiment of the present invention there is provided a compound according to formula I wherein X¹ is S, X² is chloro, bromo, difluoromethyl or cyano, R¹ is fluoro, R² is methyl, ethyl, methoxy, chloro or bromo, R³ is chloro, bromo or methyl and R⁴ is SO₂NHR^{5a}R^{6a} or COX⁴.

In a ninth embodiment of the present invention there is provided a compound according to formula I wherein X² is difluoromethyl.

In a tenth embodiment of the present invention there is provided a compound according to formula I wherein X² is difluoromethyl and R⁴ is SO₂NHR^{5a}R^{6a} or COX⁴.

In an eleventh embodiment of the present invention there is provided a compound according to formula I wherein X¹ is O, X² is difluoromethyl, R¹ is fluoro, R² is methyl, ethyl, methoxy, chloro or bromo, R³ is chloro, bromo or methyl and R⁴ is SO₂NHR^{5a}R^{6a}.

In a twelfth embodiment of the present invention there is provided a compound according to formula I wherein X¹ is O, R¹ is fluoro, R³ is chloro, bromo, or methyl, and R⁴ is —C≡CC(Me)₂R⁸.

In a thirteenth embodiment of the present invention there is provided a compound according to formula I wherein X¹ is O, R¹ is fluoro, R³ is chloro, bromo, or methyl, R⁴ is —C≡CC(Me)₂R⁸ and R⁸ is CO₂H.

In a fourteenth embodiment of the present invention there is provided a compound according to formula I wherein X¹ is O, R¹ is fluoro, R³ is chloro, bromo, or methyl, R⁴ is —C≡CC(Me)₂R⁸ and R⁸ is R⁸ is NR^{5c}R^{5d}.

In a fifteenth of the present invention there is provided a compound according to formula I wherein X¹ is O, R¹ is fluoro, R³ is chloro, bromo, or methyl, and R⁴ is A1 or A2.

In a sixteenth embodiment of the present invention there is provided a compound according to formula I wherein X¹ is O, X² is difluoromethyl, R¹ is fluoro, R² is methyl, ethyl, methoxy, chloro or bromo, R³ is chloro, bromo or methyl and R⁴ is CONR^{5b}R^{6b}. In a seventeenth embodiment of the present invention there is provided a compound according to formula I wherein the compound is provided in TABLE 1.

In eighteenth embodiment of the present invention there is provided a method for treating an HIV infection, or preventing an HIV infection, or treating AIDS or ARC, comprising administering to a host in need thereof a therapeutically effective amount of compound according to formula I wherein R¹, R², R³, R⁴, R^{5a}, R^{5b}, R^{5c}, R^{5d}, R^{6a}, R^{6b}R^{6c}, R^{6d}, R⁷, R⁸, R^{9a}, R^{9b}, R^{9c}, R^{9d}, R¹⁰, X¹, X², X³, X⁴, r and p are as defined herein above.

In nineteenth embodiment of the present invention there is provided a method for treating an HIV infection, or preventing an HIV infection, or treating AIDS or ARC, comprising administering to a host in need thereof a therapeutically effective amount of a compound according to claim 2.

In another embodiment of the present invention there is provided a method for treating an HIV infection, or preventing an HIV infection, or treating AIDS or ARC, comprising administering to a host in need thereof a therapeutically effective amount of a compound according to claim 12.

In another embodiment of the present invention there is provided a method for treating an HIV infection, or preventing an HIV infection, or treating AIDS or ARC, comprising administering to a host in need thereof a therapeutically effective amount of a compound according to claim 15.

In a twentieth embodiment of the present invention there is provided a method for treating an HIV infection, or preventing an HIV infection, or treating AIDS or ARC, comprising co-administering to a host in need thereof a therapeutically effective amount of compound according to formula I wherein R¹, R², R³, R⁴, R^{5a}, R^{5b}, R^{5c}, R^{5d}, R^{6a}, R^{6b}, R^{6c}, R^{6d}, R⁷, R⁸, R^{9a}, R^{9b}, R^{9c}, R^{9d}, R¹⁰, X¹, X², X³, X⁴, r and p are as defined herein above. and at least one compound selected from the group consisting of HIV protease inhibitors, nucleoside reverse transcriptase inhibitors, non-nucleoside reverse transcriptase inhibitors, CCR5 antagonists and viral fusion inhibitors.

In still another embodiment of the present invention there is provided a method for treating an HIV infection, or preventing an HIV infection, or treating AIDS or ARC, comprising co-administering to a host in need thereof a therapeutically effective amount of a compound according to claim 2 and at least one compound selected from the group consisting of HIV protease inhibitors, nucleoside reverse transcriptase inhibitors, non-nucleoside reverse transcriptase inhibitors, CCR5 antagonists and viral fusion inhibitors.

In still another embodiment of the present invention there is provided a method for treating an HIV infection, or preventing an HIV infection, or treating AIDS or ARC, comprising co-administering to a host in need thereof a therapeutically effective amount of a compound according to claim 12 and at least one compound selected from the group consisting of HIV protease inhibitors, nucleoside reverse transcriptase inhibitors, non-nucleoside reverse transcriptase inhibitors, CCR5 antagonists and viral fusion inhibitors.

In still another embodiment of the present invention there is provided a method for treating an HIV infection, or preventing an HIV infection, or treating AIDS or ARC, comprising co-administering to a host in need thereof a therapeutically effective amount of a compound according to claim 15 and at least one compound selected from the group consisting of HIV protease inhibitors, nucleoside reverse transcriptase inhibitors, non-nucleoside reverse transcriptase inhibitors, CCR5 antagonists and viral fusion inhibitors.

In a twenty-first embodiment of the present invention there is provided a method for treating an HIV infection, or preventing an HIV infection, or treating AIDS or ARC, comprising co-administering to a host in need thereof a therapeutically effective amount of compound according to formula I wherein R¹, R², R³, R⁴, R^{5a}, R^{5b}, R^{5c}, R^{5d}, R^{6a}, R^{6b}, R^{6c}, R^{6d}, R⁷, R⁸, R^{9a}, R^{9b}, R^{1c}, R^{9d}, R¹⁰, X¹, X², X³, X⁴, r and p are as defined herein above. and at least one compound selected from the group consisting of zidovudine, lamivudine, didanosine, zalcitabine, stavudine, rescriptor, sustiva, viramune, efavirenz, nevirapine, saquinavir, ritonavir, nelfinavir, indinavir, amprenavir, lopinavir and enfuvirtide.

In still another embodiment of the present invention there is provided a method for treating an HIV infection, or preventing an HIV infection, or treating AIDS or ARC, comprising co-administering to a host in need thereof a therapeutically effective amount of a compound according to claim 2 and at least one compound selected from the group consisting of zidovudine, lamivudine, didanosine, zalcitabine, stavudine, rescriptor, sustiva, viramune, efavirenz, nevirapine, saquinavir, ritonavir, nelfinavir, indinavir, amprenavir, lopinavir and enfuvirtide.

In yet another embodiment of the present invention there is provided a method for treating an HIV infection, or preventing an HIV infection, or treating AIDS or ARC, comprising co-administering to a host in need thereof a therapeutically effective amount of a compound according to claim 12 and at least one compound selected from the group consisting of zidovudine, lamivudine, didanosine, zalcitabine, stavudine, rescriptor, sustiva, viramune, efavirenz, nevirapine, saquinavir, ritonavir, nelfinavir, indinavir, amprenavir, lopinavir and enfuvirtide.

In yet another embodiment of the present invention there is provided a method for treating an HIV infection, or preventing an HIV infection, or treating AIDS or ARC, comprising co-administering to a host in need thereof a therapeutically effective amount of a compound according to claim 15 and at least one compound selected from the group consisting of zidovudine, lamivudine, didanosine, zalcitabine, stavudine, rescriptor, sustiva, viramune, efavirenz, nevirapine, saquinavir, ritonavir, nelfinavir, indinavir, amprenavir, lopinavir and enfuvirtide.

In a twenty-second embodiment of the present invention there is provided a method for inhibiting HIV reverse transcriptase in a host infected with HIV comprising administering to the host a therapeutically effective amount of compound according to formula I wherein R¹, R², R³, R⁴, R^{5a}, R^{5b}, R^{5c}, R^{5d}, R^{6a}, R^{6b}, R^{6c}, R^{6d}, R⁷, R⁸, R^{9a}, R^{9b}, R^{9c}, R^{9d}, R¹⁰, X¹, X², X³, X⁴, r and p are as defined herein above.

In twenty-third of the present invention there is provided a method for inhibiting HIV reverse transcriptase in a host infected with HIV comprising administering to the host a therapeutically effective amount of a compound according to claim 2.

In still another embodiment of the present invention there is provided a method for inhibiting HIV reverse transcriptase in a host infected with HIV comprising administering to the host a therapeutically effective amount of a compound according to claim 12.

In yet another embodiment of the present invention there is provided a method for inhibiting HIV reverse transcriptase in a host infected with HIV comprising administering to the host a therapeutically effective amount of a compound according to claim 15.

In a twenty-fourth embodiment of the present invention there is provided a method for inhibiting HIV reverse transcriptase with at least one mutation compared to wild type HIV reverse transcriptase in a host infected with HIV comprising administering to a host in need thereof a therapeutically effective amount of compound according to formula I wherein R¹, R², R³, R⁴, R^{5a}, R^{5b}, R^{5c}, R^{5d}, R^{6a}, R^{6b}, R^{6c}, R^{6d}, R⁷, R⁸, R^{9a}, R^{9b}, R^{9c}, R^{9d}, R¹⁰, X¹, X², X³, X⁴, r and p are as defined herein above.

In still another embodiment of the present invention there is provided a method for inhibiting HIV reverse transcriptase with at least one mutation compared to wild type HIV reverse transcriptase in a host infected with HIV comprising administering to a host in need thereof a therapeutically effective amount of a compound according to claim 2.

In still another embodiment of the present invention there is provided a method for inhibiting HIV reverse transcriptase with at least one mutation compared to wild type HIV reverse transcriptase in a host infected with HIV comprising administering to a host in need thereof a therapeutically effective amount of a compound according to claim 12.

In still another embodiment of the present invention there is provided a method for inhibiting HIV reverse transcriptase with at least one mutation compared to wild type HIV reverse transcriptase in a host infected with HIV comprising administering to a host in need thereof a therapeutically effective amount of a compound according to claim 15.

In a twenty-fifth embodiment of the present invention there is provided a method for inhibiting HIV reverse transcriptase in a host infected with strain of HIV exhibiting reduced susceptibility to efavirenz, nevirapine or delavirdine comprising administering to the host a therapeutically effective amount of compound according to formula I wherein R¹, R², R³, R⁴, R^{5a}, R^{5b}, R^{5c}, R^{5d}, R^{6a}, R^{6b}, R^{6c}, R^{6d}, R⁷, R⁸, R^{9a}, R^{9b}, R^{9c}, R^{9d}, R¹⁰, X¹, X², X³, X⁴, r and p are as defined herein above.

In yet still another embodiment of the present invention there is provided a method for inhibiting HIV reverse transcriptase in a host infected with strain of HIV exhibiting reduced susceptibility to efavirenz, nevirapine or delavirdine comprising administering to the host a therapeutically effective amount of a compound according to claim 2.

In yet still another embodiment of the present invention there is provided a method for inhibiting HIV reverse transcriptase in a host infected with strain of HIV exhibiting reduced susceptibility to efavirenz, nevirapine or delavirdine comprising administering to the host a therapeutically effective amount of a compound according to claim 12.

In yet still another embodiment of the present invention there is provided a method for inhibiting HIV reverse transcriptase in a host infected with strain of HIV exhibiting reduced susceptibility to efavirenz, nevirapine or delavirdine comprising administering to the host a therapeutically effective amount of a compound according to claim 15.

In an embodiment of the present invention there is provided a pharmaceutical composition for treating an HIV infection, or preventing an HIV infection, or treating AIDS or ARC, comprising a therapeutically effective quantity of compound according to formula I wherein R¹, R², R³, R⁴, R^{5a}, R^{5b}, R^{5c}, R^{5d}, R^{6a}, R^{6b}, R^{6c}, R^{6d}, R⁷, R⁸, R^{9a}, R^{9b}, R^{9c}, R^{9d}, R¹⁰, X¹, X², X³, X⁴, r and p are as defined herein above and at least one carrier, excipient or diluent.

In an embodiment of the present invention there is provided a pharmaceutical composition for treating an HIV infection, or preventing an HIV infection, or treating AIDS or ARC, comprising a therapeutically effective quantity of a compound according to claim 2 and at least one carrier, excipient or diluent.

In a twenty-seventh embodiment of the present invention there is provided a process for the preparation of a compound according to formula I which process comprises the steps of:

(i) condensing the salt of a substituted 3-cyanophenol II with 2,3,4-trifluoronitrobenzene to afford a biaryl ether III;

(ii) reacting III with benzaldoxime and base in an inert solvent to afford phenol IV;

(iii) alkylating the phenol IV with an alkyl bromoacetate salt or an equivalent acetic acid synthetic equivalent to afford V;

(iv) converting the nitro group in V into a chloride or bromide VI (X=Cl or Br) by the three step sequence of reduction of the nitro to an amine, diazotization of the amine and displacement of the diazo group with chloride or bromide and optionally displacing the bromide thus formed with a dialkylzinc in the presence of a palladium catalyst to afford VI (X=alkyl);

(iv) converting the ester VI into the corresponding 4-sulfamoyl-anilide VII (R⁴=SO₂NHR^{5a}R^{6a}) or 4 carbamoyl-anilide VII (R⁴=CONR^{5b}R^{6b}).

In another embodiment of the present invention there is provided a compound select from I-1 to I-32 of TABLE 1.

### DEFINITIONS

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

It is contemplated that the definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” and the like. When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero)aryl or (het)aryl refers to either an aryl or a heteroaryl group.

The term “optional” or “optionally” as used herein means that a subsequently described event or circumstance may, but need not, occur, and that the description includes instances where the event or circumstance occurs and instances in which it does not. For example, “optionally substituted” means that the optionally substituted moiety may incorporate a hydrogen or a substituent.

The phrase “optional bond” means that the bond may or may not be present, and that the description includes single, double, or triple bonds. If a substituent is designated to be a “bond” or “absent”, the atoms linked to the substituents are then directly connected.

When any variable (e.g., R¹, R^{4a}, Ar, X¹ or Het) occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

Unless expressly stated to the contrary, all ranges cited herein are inclusive. For example, a heterocyclic ring described as containing “1 to 4 heteroatoms” means the ring can contain 1, 2, 3 or 4 heteroatoms. It is also to be understood that any range cited herein includes within its scope all of the subranges within that range. Thus, for example, an aryl or a heteroaryl described as optionally substituted with “from 1 to 5 substituents” is intended to include as aspects thereof, any aryl optionally substituted with 1 to 4 substituents, 1 to 3 substituents, 1 to 2 substituents, 2 to 5 substituents, 2 to 4 substituents, 2 to 3 substituents, 3 to 5 substituents, 3 to 4 substituents, 4 to 5 substituents, 1 substituent, 2 substituents, 3 substituents, 4 substituents, and 5 substituents.

The symbol “*” at the end of a bond or “-------” drawn through a bond each refer to the point of attachment of a functional group or other chemical moiety to the rest of the molecule of which it is a part. Thus, for example:

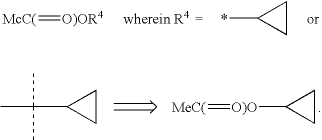

The term “acyl” as used herein denotes a group of formula —C(═O)R wherein R is hydrogen or lower alkyl as defined herein. C₁₋₃ acyl denotes an acyl group as defied herein wherein R is C₁₋₃ alkyl.

The term “alkyl” as used herein denotes an unbranched or branched chain, saturated, monovalent hydrocarbon residue containing 1 to 10 carbon atoms. The term “lower alkyl” denotes a straight or branched chain hydrocarbon residue containing 1 to 6 carbon atoms. “C₁₋₁₀ alkyl” as used herein refers to an alkyl composed of 1 to 10 carbons.

The terms “amino”, “alkylamino” and “dialkylamino” as used herein refer to —NH₂, —NHR and —NR₂ respectively and R is alkyl as defined above. The two alkyl groups attached to a nitrogen in a dialkyl moiety can be the same or different. The terms “aminoalkyl”, “alkylaminoalkyl” and “dialkylaminoalkyl” as used herein refer to NH₂(CH₂)n—, RHN(CH₂)n—, and R₂N(CH₂)n—respectively wherein n is 1 to 10 and R is alkyl as defined above. “C₁₋₆ alkylamino” as used herein refers to an aminoalkyl wherein alkyl is C₁₋₆. The term “phenylamino” as used herein refers to —NHPh wherein Ph represents an optionally substituted phenyl group.

The term “cycloalkyl” as used herein denotes a saturated carbocyclic ring containing 3 to 8 carbon atoms, i.e. cyclopropyl, cyclobutyl, cyclopentyl, cyclohexyl, cycloheptyl or cyclooctyl. “C₃₋₅ cycloalkyl” as used herein refers to a cycloalkyl composed of 3 to 5 carbons in the carbocyclic ring.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” refers to an-O-alkyl wherein alkyl is C1-10.

The term “cyano” as used herein refers to a carbon linked to a nitrogen by a triple bond, i.e., —C—N. The term “nitro” as used herein refers to a group —NO2.

The term “haloalkyl” as used herein denotes an unbranched or branched chain alkyl group as defined above wherein 1, 2, 3 or more hydrogen atoms are substituted by a halogen. “C₁₋₃ haloalkyl” as used herein refers to a haloalkyl composed of 1 to 3 carbons and I-8 halogen substituents. Examples are 1-fluoromethyl, 1-chloromethyl, 1-bromomethyl, 1-iodomethyl, trifluoromethyl, trichloromethyl, tribromomethyl, triiodomethyl, 1-fluoroethyl, 1-chloroethyl, 1-bromoethyl, 1-iodoethyl, 2-fluoroethyl, 2-chloroethyl, 2-bromoethyl, 2-iodoethyl, difluoromethyl, 2,2-dichloroethyl, 3-bromopropyl or 2,2,2-trifluoroethyl.

The term “haloalkoxy” as used herein means a —O-haloalkyl group wherein haloalkyl is defined herein.

The term “halogen” or “halo” as used herein means fluorine, chlorine, bromine, or iodine.

The terms “hydroxyalkyl” and “alkoxyalkyl” as used herein denotes alkyl radical as herein defined wherein one to three hydrogen atoms on different carbon atoms is/are replaced by hydroxyl or alkoxy groups respectively. C₁₋₆ hydroxyalkyl refers to a C₁₋₆ alkyl group as herein defined wherein one to three hydrogen atoms on different carbon atoms is/are replaced by a hydroxyl groups.

The term “C₁₋₆ carboxyalkyl” as used herein refers to a C₁₋₆ alkyl group as herein defined wherein one or two hydrogen atoms on different carbon atoms is/are replaced by a hydroxyl groups. The group NR^{a}R^{b} as used in claim 1 where R^{a} is a carboxyalkyl group which includes, but is not limited to, the natural amino acids glycine, alanine, valine, leucine and isoleucine.

The terms “azetidine”, “pyrrolidine”, “piperidine” and “azepine” refer to a 4-, 5-, 6- or 7-membered cycloalkane respectively wherein one carbon atom is replaced by a nitrogen atom.

The term “aryl” as used herein denotes a phenyl ring which can optionally be substituted with one or more, preferably one or three substituents independently selected from hydroxy, thio, cyano, alkyl, alkoxy, lower haloalkoxy, alkylthio, halogen, haloalkyl, hydroxyalkyl, nitro, alkoxycarbonyl, amino, alkylamino, dialkylamino, aminoalkyl, alkylaminoalkyl, and dialkylaminoalkyl, alkylsulfonyl, arylsulfinyl, alkylaminosulfonyl, arylaminosulfonyl, alkylsulfonylamino, arylsulfonylamino, carbamoyl, alkylcarbamoyl and dialkylcarbamoyl, arylcarbamoyl, alkylcarbonylamino, arylcarbonylamino, unless otherwise indicated. Alternatively two adjacent atoms of the aryl ring may be substituted with a methylenedioxy or ethylenedioxy group. The term “aryloxy” as used herein denotes an optionally substituted phenol.

The term “inert organic solvent” or “inert solvent” means the solvent is inert under the conditions of the reaction being described in conjunction therewith. In the case of the reaction of benzaldoxime with base an inert solvent is one which neither has an acidic proton nor will react with trifluoronitrobenzene. Examples on inert solvents include ethereal solvents and hydrocarbons. The term “base” refers to an organic or inorganic base of sufficient strength to deprotonate the phenol II. Examples of such bases are numerous and well know in the art.

An acetic acid synthetic equivalent of an alkyl bromoacetate is an acetic acid derivative with a leaving group on the α-carbon which is capable of being displaced by a phenolate salt. While the reaction is exemplified herein with ethyl bromoacetate other esters could be utilized in analogously. The ester also could be replaced with an amide including the anilide derivatives described herein.

The term “wild type” as used herein refers to the HIV virus strain which possesses the dominant genotype which naturally occurs in the normal population which has not been exposed to reverse transcriptase inhibitors. The term “wild type reverse transcriptase” used herein has refers to the reverse transcriptase expressed by the wild type strain which has been sequenced and deposited in the SwissProt database with an accession number P03366.

The term “reduced susceptibility” as used herein refers to about a 10 fold, or greater, change in sensitivity of a particular viral isolate compared to the sensitivity exhibited by the wild type virus in the same experimental system.

The term “nucleoside and nucleotide reverse transcriptase inhibitors” (“NRTI”s) as used herein means nucleosides and nucleotides and analogues thereof that inhibit the activity of HIV-1 reverse transcriptase, the enzyme which catalyzes the conversion of viral genomic HIV-1 RNA into proviral HIV-1 DNA. Recent progress in development of RTI and PI inhibitors have been reviewed: F. M. Uckun and O. J. D'Cruz, Exp. Opin. Ther. Pat. 2006 16:265-293; L. Menendez-Arias, Eur. Pharmacother. 2006 94-96 and S. Rusconi and O. Vigano, Future Drugs 2006 3(1):79-88.

Typical suitable NRTIs include zidovudine (AZT; RETROVIR®); didanosine (ddI; VIDEX®); zalcitabine (ddC; HIVID®); stavudine (d4T; ZERIT®); lamivudine (3TC; EPIVIR®); abacavir (ZIAGEN®); adefovir dipivoxil [bis(POM)-PMEA; PREVON®]; lobucavir (BMS-180194), a nucleoside reverse transcriptase inhibitor disclosed in EP-0358154 and EP-0736533; BCH-10652, a reverse transcriptase inhibitor (in the form of a racemic mixture of BCH-10618 and BCH-10619) under development by Biochem Pharma; emitricitabine [(−)-FTC] in development by Triangle Pharmaceuticals; β-L-FD4 (also called β-L-D4C and named β-L-2′,3′-dicleoxy-5-fluoro-cytidene) licensed Vion Pharmaceuticals; DAPD, the purine nucleoside, (−)-β-D-2,6-diamino-purine dioxolane disclosed in EP-0656778 and licensed to Triangle Pharmaceuticals; and lodenosine (FddA), 9-(2,3-dideoxy-2-fluoro-β-D-threo-pentofuranosyl)adenine, an acid stable purine-based reverse transcriptase inhibitor under development by U.S. Bioscience Inc.

Typical suitable NNRTIs include nevirapine (BI-RG-587; VIRAMUNE®); delaviradine (BHAP, U-90152; RESCRIPTOR®); efavirenz (DMP-266; SUSTIVA®); PNU-142721, a furopyridine-thio-pyrimidine under development by Pfizer; AG-1549 (formerly Shionogi # S-1153); 5-(3,5-dichlorophenyl)-thio-4-isopropyl-1-(4-pyridyl)methyl-1H-imidazol-2-ylmethyl carbonate disclosed in WO 96/10019; MKC-442 (1-(ethoxy-methyl)-5-(1-methylethyl)-6-(phenylmethyl)-(2,4(1H, 3H)-pyrimidinedione); and (+)-calanolide A (NSC-675451) and B, coumarin derivatives disclosed in U.S. Pat. No. 5,489,697.

The term “protease inhibitor” (“PI”) as used herein means inhibitors of the HIV-1 protease, an enzyme required for the proteolytic cleavage of viral polyprotein precursors (e.g., viral GAG and GAG Pol polyproteins), into the individual functional proteins found in infectious HIV-1. HIV protease inhibitors include compounds having a peptidomimetic structure, high molecular weight (7600 daltons) and substantial peptide character, e.g. CRIXIVAN® as well as nonpeptide protease inhibitors e.g., VIRACEPT®.

Typical suitable PIs include saquinavir (Ro 31-8959; INVIRASE®; FORTOVASE®); ritonavir (ABT-538; NORVIR®); indinavir (MK-639; CRIXIVAN®); nelfnavir (AG-1343; VIRACEPT®); amprenavir (141W94; AGENERASE®); TMC114 (darunavir, PREZISTA®); lasinavir (BMS-234475); DMP-450, a cyclic urea under development by Triangle Pharmaceuticals; BMS-2322623, an azapeptide under development by Bristol-Myers Squibb as a 2nd-generation HIV-1 PI; ABT-378 under development by Abbott; and AG-1549 an imidazole carbamate under development by Agouron Pharmaceuticals, Inc.

Pentafuside (FUZEON®) a 36-amino acid synthetic peptide that inhibits fusion of HIV-1 to target membranes. Pentafuside (3-100 mg/day) is given as a continuous sc infusion or injection together with efavirenz and 2 PI's to HIV-1 positive patients refractory to a triple combination therapy; use of 100 mg/day is preferred. FUZEON binds to GP41 on the viral coating and prevents the creation of an entry pore for the capsid of the virus keeping it out of the cell.

HIV-1 infects cells of the monocyte-macrophage lineage and helper T-cell lymphocytes by exploiting a high affinity interaction of the viral enveloped glycoprotein (Env) with the CD-4 antigen. The CD-4 antigen was found to be a necessary, but not sufficient requirement for cell entry and at least one other surface protein was required to infect the cells (E. A. Berger et al., Ann. Rev. Immunol. 1999 17:657-700). Two chemokine receptors, either the CCR5 or the CXCR4 receptor were subsequently found to be co-receptors along with CD4 which are required for infection of cells by the human immunodeficiency virus (HIV). Antagonists of CCR5 binding have been sought to prevent viral fusion. Maraviroc (Pfizer) is a CCR5 antagonists which is nearing approval by the FDA. Vicriviroc (Schering) by Pfizer is in late development stage. Numerous other companies have research programs in various discovery and development stages (see, e.g. A. Palani and J. R. Tagat, J. Med. Chem. 2006 49(10):2851-2857, P. Biswas et al. Expert. Opin. Investig. Drugs 2006 15(5):451-464; W. Kazmierski et al. Biorg Med. Chem. 2003 11:2663-76). The CCR5 antagonists which reach the marketplace while likely be useful in combination with NNRTIs, NRTIs and PIs.

Attachment Inhibitors effectively block interaction between viral envelope proteins and chemokine receptors or CD40 protein._TNX-355 is a humanized IgG4 monoclonal antibody that binds to a conformational epitope on domain 2 of CD4. (L. C. Burkly et al., J. Immunol. 1992 149:1779-87) TNX-355 can inhibit viral attachment of CCR5-, CXCR4- and dual/mixed tropic HIV-1 strains. (E. Godofsky et al., In Vitro Activity of the Humanized Anti-CD4 Monoclonal Antibody, TNX-355, against CCR5, CXCR4, and Dual-Tropic Isolates and Synergy with Enfuvirtide, 45th Annual Interscience Conference on Antimicrobial Agents and Chemotherapy (ICAAC). Dec. 16-19, 2005, Washington D.C. Abstract # 3844; D. Norris et al. TNX-355 in Combination with Optimized Background Regime (OBR) Exhibits Greater Antiviral Activity than OBR Alone in HIV-Treatment Experienced Patients, 45th Annual Interscience Conference on Antimicrobial Agents and Chemotherapy (ICAAC). Dec. 16-19, 2005, Washington D.C. Abstract # 4020.)

Macromolecular therapeutics including antibodies, soluble receptors and biologically active fragments thereof have become an increasingly important adjunct to conventional low molecular weight drugs. (O. H. Brekke and I. Sandlie Nature Review Drug Discov. 2003 2:52-62; A. M. Reichert Nature Biotech. 2001 19:819-821) Antibodies with high specificity and affinity can be targeted at extra-cellular proteins essential for viral cell fusion. CD4, CCR5 and CXCR4 have been targets for antibodies which inhibit viral fusion.

V. Roschke et al. (Characterization of a Panel of Novel Human Monoclonal Antibodies that Specifically Antagonize CCR5 and Block HIV-1 Entry, 44th Annual Interscience Conference on Antimicrobial Agents and Chemotherapy (ICAAC). Oct. 29, 2004, Washington D.C. Abstract # 2871) have disclosed monoclonal antibodies which bind to the CCR5 receptor and inhibit HIV entry into cells expressing the CCR5 receptor. L. Wu and C. R MacKay disclose in U.S. Ser. No. 09/870,932 filed May 30, 2001 disclose monoclonal antibodies 5C7 and 2D7 which bind to the CCR5 receptor in a manner capable of inhibiting HIV infection of a cell. W. C. Olsen et al. (J. Virol. 1999 73(5):4145-4155) disclose monoclonal antibodies capable of inhibiting (i) HIV-1 cell entry, (ii) HIV-1 envelope-mediated membrane fusion, (iii) gp120 binding to CCR5 and (iv) CC-chemokine activity. Synergism between the anti-CCR5 antibody Pro140 and a low molecular weight CCR5 antagonists have been disclosed by Murga et al. (3rd IAS Conference on HIV Pathogenesis and Treatment, Abstract TuOa.02.06. Jul. 24-27, 2005, Rio de Janeiro, Brazil) Anti-CCR5 antibodies have been isolated which inhibit HIV-1 cell entry also have been disclosed by M. Brandt et al. in U.S. Ser. No. 11/394,439 filed Mar. 31, 2006.

Other antiviral agents which may be useful in HIV therapy include hydroxyurea, ribavirin, IL-2, IL-12, pentafuside. Hydroyurea (Droxia), a ribonucleoside triphosphate reductase inhibitor, the enzyme involved in the activation of T-cells, was discovered at the NCI and is under development by Bristol-Myers Squibb; in preclinical studies, it was shown to have a synergistic effect on the activity of didanosine and has been studied with stavudine. IL-2 is disclosed in Ajinomoto EP-0142268, Takeda EP-0176299, and Chiron U.S. Pat. Nos. RE 33,653, 4,530,787, 4,569,790, 4,604,377, 4,748,234, 4,752,585, and 4,949,314, and is available under the PROLEUKIN® (aldesleukin) from Chiron Corp. as a lyophilized powder for IV infusion or sc administration. IL-12 is disclosed in WO96/25171 and is available from Roche and Wyeth Pharmaceuticals. Ribavirin, 1-β-D-ribofuranosyl-1H-1,2,4-triazole-3-carboxamide, is described in U.S. Pat. No. 4,211,771 and is available from ICN Pharmaceuticals.

Abbreviations used in this application include: acetyl (Ac), acetic acid (HOAc), azo-bis-isobutyrylnitrile (AIBN), 1-N-hydroxybenzotriazole (HOBt), atmospheres (Atm), high pressure liquid chromatography (HPLC), 9-borabicyclo[3.3.1]nonane (9-BBN or BBN), methyl (Me), tert-butoxycarbonyl (Boc), acetonitrile (MeCN), di-tert-butyl pyrocarbonate or boc anhydride (BOC₂O), 1-(3-dimethylaminopropyl)-3-ethylcarbodiimide hydrochloride (EDCI), benzyl (Bn), m-chloroperbenzoic acid (MCPBA), butyl (Bu), methanol (MeOH), benzyloxycarbonyl (cbz or Z), melting point (mp), carbonyl diimidazole (CDI), MeSO₂— (mesyl or Ms), 1,4-diazabicyclo[2.2.2]octane (DABCO), mass spectrum (ms) diethylaminosulfur trifluoride (DAST), methyl t-butyl ether (MTBE), dibenzylideneacetone (Dba), N-carboxyanhydride (NCA), 1,5-diazabicyclo[4.3.0]non-5-ene (DBN), N-bromosuccinimide (NBS), N-chlorosuccinimide (NCS),1,8-diazabicyclo[5.4.0]undec-7-ene (DBU), N-methylmorpholine (NMM), N-methylpyrrolidone (NMP), 1,2-dichloroethane (DCE), pyridinium chlorochromate (PCC), N,N′-dicyclohexylcarbodiimide (DCC), pyridinium dichromate (PDC), dichloromethane (DCM), propyl (Pr), diethyl azodicarboxylate (DEAD), phenyl (Ph), di-iso-propylazodicarboxylate, DIAD, pounds per square inch (psi), di-iso-propylethylamine (DIPEA), pyridine (pyr), di-iso-butylaluminumhydride, DIBAL-H, room temperature, rt or RT, N,N-dimethyl acetamide (DMA), tert-butyldimethylsilyl or t-BuMe₂Si, (TBDMS), 4-N,N-dimethylaminopyridine (DMAP), triethylamine (Et₃N or TEA), N,N-dimethylformamide (DMF), triflate or CF₃SO₂— (Tf), dimethyl sulfoxide (DMSO), trifluoroacetic acid (TFA), 1,1′-bis-(diphenylphosphino)ethane (dppe), 2,2,6,6-tetramethylheptane-2,6-dione (TMHD), 1,1′-bis-(diphenylphosphino)ferrocene (dppf), thin layer chromatography (TLC), ethyl acetate (EtOAc), tetrahydrofuran (THF), diethyl ether (Et₂O), trimethylsilyl or Me₃Si (TMS), ethyl (Et), p-toluenesulfonic acid monohydrate (TSOH or pTsOH), lithium hexamethyl disilazane (LiHMDS),4-Me-C₆H₄SO₂— or tosyl (Ts), iso-propyl (i-Pr), N-urethane-N-carboxyanhydride (UNCA), ethanol (EtOH). Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford.).

### COMPOUNDS AND PREPARATION

Compounds of the present invention can be made by a variety of methods depicted in the illustrative synthetic reaction schemes shown and described below. The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, Volumes 1-21; R. C. LaRock, Comprehensive Organic Transformations, 2^{nd} edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, Volumes 1-40. The following synthetic reaction schemes are merely illustrative of some methods by which the compounds of the present invention can be synthesized, and various modifications to these synthetic reaction schemes can be made and will be suggested to one skilled in the art having referred to the disclosure contained in this Application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, e.g., about 20° C.

Some compounds in following schemes are depicted with generalized substituents; however, one skilled in the art will immediately appreciate that the nature of the R groups can varied to afford the various compounds contemplated in this invention. Moreover, the reaction conditions are exemplary and alternative conditions are well known. The reaction sequences in the following examples are not meant to limit the scope of the invention as set forth in the claims.

Examples of representative compounds encompassed by, and within the scope of, the present invention are provided in the following Table. These examples and preparations which follow are provided to enable those skilled in the art to more clearly understand and to practice the present invention. They should not be considered as limiting the scope of the invention, but merely as being illustrative and representative thereof.

In general, the nomenclature used in this Application is based on AUTONOM™ v. 4.0, a Beilstein Institute computerized system for the generation of IUPAC systematic nomenclature. If there is a discrepancy between a depicted structure and a name given that structure, the depicted structure is to be accorded more weight.

TABLE 1

| | 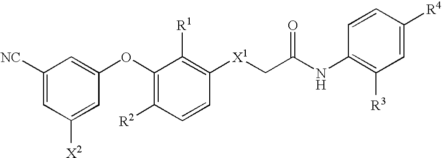 | | | | | | | MS | HIV-RT |
|---|---|---|---|---|---|---|---|---|---|
| Cpd. No. | R¹ | R² | R³ | R⁴ | X¹ | X² | MP | [M − 1] | IC₅₀ (μM) |
| I-1 | F | Cl | Cl | SO₂NH₂ | O | Cl | 233.3- | 542 | 0.0162 |
| | | | | | | | 247.6 | | |
| I-2 | F | Cl | Me | SO₂NH₂ | O | Cl | 209.9- | 522 | 0.0128 |
| | | | | | | | 212.0 | | |
| I-3 | F | Cl | Br | SO₂NH₂ | O | Cl | | 568 | >10 |
| I-4 | F | Br | Me | SO₂NH₂ | O | Cl | 226.0- | 566 | 0.0065 |
| | | | | | | | 228.8 | | |
| I-5 | F | Br | Cl | SO₂NH₂ | O | Cl | 250.9- | 586 | 0.0187 |
| | | | | | | | 252.3 | | |
| I-6 | F | Et | Me | SO₂NH₂ | O | Cl | 188.0- | 516 | 0.0084 |
| | | | | | | | 189.6 | | |
| I-7 | F | Et | Cl | SO₂NH₂ | O | Cl | | 536 | >10 |
| I-8 | F | c-C₃H₅ | Cl | SO₂NH₂ | O | Cl | 210.0- | 548 | 0.0183 |
| | | | | | | | 211.0 | | |
| I-9 | F | c-C₃H₅ | Me | SO₂NH₂ | O | Cl | 185.5- | 528 | 0.0077 |
| | | | | | | | 187.0 | | |
| I-10 | H | Cl | Cl | SO₂NH₂ | O | Cl | 217.0- | 524 | 0.0074 |
| | | | | | | | 219.0 | | |
| I-11 | F | Cl | Me | SO₂NH₂ | O | MeO | 122.0- | 518 | 0.0031 |
| | | | | | | | 123.2 | | |
| I-12 | F | Cl | Cl | SO₂NH₂ | O | MeO | 211.0- | 538 | 0.0082 |
| | | | | | | | 212.0 | | |
| I-13 | F | Cl | Me | SO₂NH₂ | O | CN | 264.5- | 513 | 0.0038 |
| | | | | | | | 265.2 | | |
| I-14 | F | Cl | Cl | SO₂NH₂ | O | CN | 258.0- | 533 | 0.0064 |
| | | | | | | | 260.0 | | |
| I-15 | H | Cl | Me | SO₂NH₂ | O | Cl | | 504 | 0.0065 |
| I-16 | F | Br | Me | SO₂NH₂ | O | MeO | 220.0- | 562 | 0.0051 |
| | | | | | | | 223.3 | | |
| I-17 | F | Br | Cl | SO₂NH₂ | O | MeO | 213.9- | 584 | 0.0126 |
| | | | | | | | 217.9 | | |
| I-18 | F | Cl | Me | SO₂NH₂ | O | Br | 234.9- | 566 | 0.0066 |
| | | | | | | | 236.6 | | |
| I-19 | F | Cl | Cl | SO₂NH₂ | O | Br | 242.2- | 586 | 0.0149 |
| | | | | | | | 244.9 | | |
| I-20 | F | Cl | Cl | SO₂NH₂ | O | Et | | 536 | 0.0317 |
| I-21 | F | Br | Cl | SO₂NH₂ | O | CN | 255.0- | 577 | 0.0191 |
| | | | | | | | 257.0 | | |
| I-22 | F | Br | Me | SO₂NH₂ | O | CN | 253.5- | 577 | 0.0191 |
| | | | | | | | 255.0 | | |
| I-23 | F | Br | Cl | SO₂N⁻COEt Na⁺ | O | Cl | 261.0- | 642 | 0.0048 |
| | | | | | | | 264.0 | | |
| I-24 | F | Br | Me | SO₂N⁻COEt Na⁺ | O | Cl | 268.1- | 622 | 0.0057 |
| | | | | | | | 270.0 | | |
| I-25 | F | Cl | Me | SO₂NH₂ | O | c-C₃H₅ | 206.5- | 528 | 0.0184 |
| | | | | | | | 207.5 | | |
| I-26 | F | Cl | Cl | SO₂NH₂ | O | c-C₃H₅ | 219.0- | 548 | 0.0665 |
| | | | | | | | 221.0 | | |
| I-27 | F | Cl | Cl | CO₂H | O | Cl | | 507 | 0.0061 |
| I-28 | F | Br | Me | CONH₂ | O | Cl | 250.6- | 532 | 0.0069 |
| | | | | | | | 252.5 | | |
| I-29 | F | Br | Cl | SO₂NH₂ | O | CHF₂ | 226.5- | | 0.0273 |
| | | | | | | | 228.3 | | |
| I-30 | F | Br | Cl | 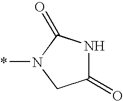 | O | Cl | 260.8-262.8 | 591[M + H] | 0.0155 |
| I-31 | F | Br | Cl | 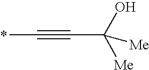 | O | Cl | | 590, 592& 594 | 0.0821 |
| I-32 | F | Cl | Cl | 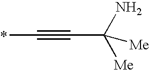 | O | Cl | 206.0-207.4 | 529 &531[M-NH2] | 0.0214 |

Compounds of the present invention are prepared (SCHEME A) from a 4-nitro-3-aryloxyphenol (18) which can prepared from 2,3,4-trifluoronitrobenzene or 2,4-dinitrobenzene by a two step process comprising nucleophilic aromatic displacement of the 2-fluoro by an appropriately substituted phenol and subsequent displacement of the 4-fluoro with benzaldehyde oxime under conditions which result in cleavage of the N—O bond (R. D. Knudsen and H. R. Snyder, J. Org. Chem. 1974 39(23):3343-3346). One skilled in the art will appreciate that the reaction can employed with a variety of phenols with diverse substitution and regiochemistry. Thioethers disclosed herein can be prepared by direct displacement of the fluorine with an alkyl thiolglycolic acid.

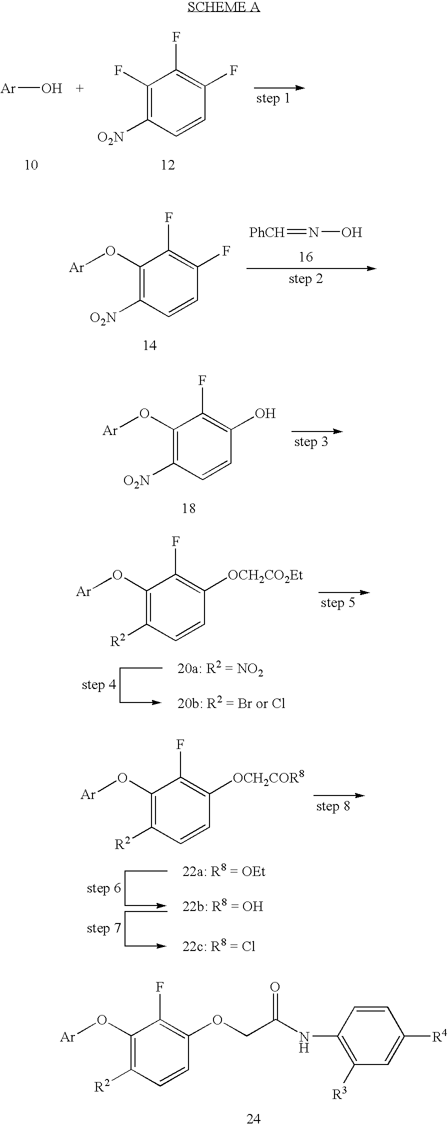

Fluoronitroaromatic compounds are known to be unusually sensitive to nucleophilic attack by soft nucleophiles. Fluorine substituents are generally significantly more labile than other halogen substituents. While hard nucleophiles like water and hydroxide fail to displace fluoride, soft nucleophiles like phenols, imidazoles, amines, thiols and some amides undergo facile displacement reactions even at room temperature (D. Boger et al., Biorg. Med. Chem. Lett. 2000 10: 1471-75; F. Terrier Nucleophilic Aromatic Displacement: The Influence of the Nitro Group VCH Publishers, New York, N.Y. 1991). In U.S. Pat. No. 5,292,967 issued Mar. 8, 1994 T. Papenfuhs et al. disclose a process for preparing 2,3-difluoro-6-nitro-phenol in good yields and high selectivity by treating 12 with an alkali metal hydroxide and an alkaline metal hydroxide. J. H. Marriott et al. (J. Chem. Soc. Perkin 12000 4265-4278) disclose the addition of alkali alkoxides to preponderantly to the para position of pentafluoro-nitro-benzene under phase-transfer conditions (DCM/aq NaOH/Bu₄N⁺HSO₄⁻/RT). 2,4-difluoro-nitro-benzene reacted non-regioselectively to afford both para and ortho displacement. The reaction of sodium methoxide with 2,3,4-trifluoronitrobenzene in methanol has been reported to afford an inseparable mixture of the corresponding 2- and 4-monomethoxy and 2,4-dimethoxy derivatives (P. M. O'Neill et al., J. Med. Chem. 1994 37:1362-70). Displacement of the ortho-fluorine of 2,4-difluoronitrobenzene by amine nucleophiles also has been reported. (W. C. Lumma, Jr. et al., J. Med. Chem. 1981 24:93-101).

Compounds of the present invention possess a variety of substituents at 4-position of the phenoxyacetic acid moiety and the nitro group can be exploited to introduce other substituents utilizing the Sandmeyer reaction. SCHEME A depicts the introduction a halogen moiety by reduction of the nitro group, diazotization of the resulting amine and displacement with halogen. When the halogen is bromine, palladium-mediated displacements permit introduction of alkyl substituents.

Reduction of the nitro group can be carried out with a variety of well-known reducing agents. For example an activated metal such as activated iron, zinc or tin (produced for example by washing iron powder with a dilute acid solution such as dilute hydrochloric acid). The reduction can also be carried out under a hydrogen atmosphere in the presence of an inert solvent in the presence of a metal effective to catalyze hydrogenation reactions such as platinum or palladium. Other reagents which have been used to reduce nitro compounds to amines include AlH₃—AlCl₃, hydrazine and a catalyst, TiCl₃, Al—NiCl₂-THF, formic acid and Pd/C and sulfides such as NaHS, (NH₄)₂S or polysulfides (i.e. the Zinn reaction). Aromatic nitro groups have been reduces with NaBH₄ or BH₃ in the presence of catalysts such as NiCl₂ and CoCl₂. Thus for example, reduction may be effected by heating the nitro group in the presence of a sufficiently activated metal such as Fe and a solvent or diluent such as H₂O and alcohol, for example MeOH or EtOH at a temperature in the range of 50 to 150° C., conveniently at about 70° C. (J. March, Advanced Organic Chemistry, John Wiley & Sons: New York, N.Y., 1992, p 1216)

Conversion of an aryl amine to an aryl halides was carried out by diazotization of the amine and displacement of the resulting diazonium group with a halide were carried out under standard conditions. Diazotization of the aryl amines is accomplished by treating the amine with nitrous acid which is commonly formed by treating a solution of the amine in dilute HCl with an aqueous solution of sodium nitrite at 0-10° C. Other mineral acids such as sulfuric acid and phosphoric acid can be used if the chloride counterion is undesirable. Diazotization of amines can be carried out in organic solvents such as HOAc, MeOH, EtOH, formamide and DMF in the presence of nitrous acid esters such as. butyl nitrite and pentyl nitrite. (K. Schank, Preparation of diazonium groups, In The chemistry of diazonium and diazo groups, Part 2; S. Patai, Ed.; John Wiley & Sons: New York, N.Y., 1978, p. 647-648). Conversion of the resulting diazonium salt to a chlorine or bromine is carried out in HCl/Cu(I) Cl or HBr/Cu(I)Br. Aryl bromide and chlorides can also be prepared from primary aromatic amines by treating the amine with tert-butyl nitrite and anhydrous CuCl₂ or CuBr₂ at 65° C. or with tert-butyl thionitrite or tert-butyl-thionitrate and CuCl₂ or CuBr₂ at RT. (J. March, Advanced Organic Chemistry, John Wiley & Sons: New York, N.Y., 1992, p 723)

Other compounds with the scope of the present invention are substituted with an alkyl or cycloalkyl group at the 4-position of the phenoxyacetic acid. Alkyl and alkenyl groups can be introduced utilizing the Negishi coupling of organozinc halides, dialkylzinc or dialkenyl zinc with haloarenes and aryl triflates is an effective means for attachment of an alkyl group to an arene (E.-I. Negishi, Acc. Chem. Res. 1982 15:340-348). The reaction is catalyzed by palladium Pd(0) and palladium is preferably ligated to a bidentate ligand including Pd(dppf)Cl₂ and Pd(dppe)Cl₂. (J. M. Herbert Tetrahedron Lett. 2004 45:817-819) Typically the reaction is run an inert aprotic solvent and common ethereal solvents include dioxane, DME and THF are suitable. The reaction is commonly run at elevated temperature. The Negishi reaction was utilized to introduce methyl and ethyl substituents.

The 4-cyclopropyl substituent is introduced in two steps by ethenyltrimethyltin mediated displacement of the bromide and cyclopropanation of the resulting olefin. The cyclopropanation was achieved Pd(OAc)₂ catalyzed cycloaddition of diazomethane. Other cyclopropanation conditions are well known in the art and could be adapted to this substrate.

Introduction of the acetic acid is readily accomplished by alkylating the phenol with commercially available alkyl haloacetates in the presence of base. Hydrolysis of the resulting ethyl ester, conversion to the acid chloride and condensation with an aniline are all performed using standard methodology

The amide may be formed by any appropriate amidation means known in the art from the corresponding esters or carboxylic acids. One way to prepare such compounds is to convert an acid to an acid chloride and then treat that compound with ammonium hydroxide or an appropriate amine. For example, the ester is treated with an alcoholic base solution such as ethanolic KOH or LiOH (in approximately a 10% molar excess) at room temperature for about 30 minutes. The solvent is removed and the residue taken up in an organic solvent such as diethyl ether, treated with a dialkyl formamide and an excess of oxalyl chloride. This is all affected at a moderately reduced temperature between about −10 to 10° C. The resulting solution is then stirred at the reduced temperature for 1-4 hours. Solvent removal provides a residue which is taken up in an inert organic solvents e.g., DCM, EtOAc, THF or toluene, cooled to about 0° C. and treated with concentrated ammonium hydroxide or an appropriate amine. Excess amine must be provided as the reaction produces HCl which forms a non-reactive ammonium salt. Alternatively a trialkyl amine or pyridine is incorporated in the reaction as a base to react with the HCl formed during the reaction. The resulting mixture is stirred at a reduced temperature for 1-4 hours. Alternatively one skilled in the art will appreciate that the amidation of an acyl halide can be carried out in an aqueous organic solvent in the presence of an alkali metal carbonate and the appropriate amine (Schotten-Bauman conditions)

Alternatively the acid may be activated with 1 equivalent of a suitable coupling agent or dehydrating agent, e.g., EDCI, CDI or DCC. Numerous additives have been identified which improve the coupling efficiency including, 1-hydroxybenzotriazole and 3-hydroxy-3,4-dihydro-4-oxo-1,2,3-benzotriazine (W. König and R. Geiger Chem. Ber. 1970 788:2024 and 2034), N-hydroxysuccinimide (E. Wunsch and F. Drees, Chem. Ber. 1966 99:110), 1-hydroxy-7-azabenzotriazole (L. A. Carpino J. Am. Chem. Soc. 1993 115:4397-4398). Protocols for dehydrative coupling have been extensively refined in the peptide synthesis art and these protocols can be used herein. These protocols have been reviewed, see e.g., M. Bodanszky, Principles of Peptide Synthesis, Springer Verlag, New York 1993; P. Lloyd-Williams and F. Albericio Chemical Methods for the Synthesis of peptides and Proteins CRC Press, Boca Raton, Fla. 1997.

L. H. Jones et al. describe the preparation of 3-chloro-5-hydroxy-benzonitrile utilized in examples 6-8 of the U.S. Pub. No. 20050004129 published Jan. 6, 2005. The preparation of other phenols which can be used to prepare compounds of the present invention can be found in the examples (infra).

### DOSAGE AND ADMINISTRATION

The compounds of the present invention may be formulated in a wide variety of oral administration dosage forms and carriers. Oral administration can be in the form of tablets, coated tablets, dragees, hard and soft gelatine capsules, solutions, emulsions, syrups, or suspensions. Compounds of the present invention are efficacious when administered by other routes of administration including continuous (intravenous drip) parenteral, intramuscular, intravenous, and suppository administration, among other routes of administration. The preferred manner of administration is generally oral using a convenient daily dosing regimen which can be adjusted according to the degree of affliction and the patient's response to the active ingredient.

A compound or compounds of the present invention, as well as their pharmaceutically useable salts, together with one or more conventional excipients, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. A typical preparation will contain from about 5% to about 95% active compound or compounds (w/w). The term “preparation” or “dosage form” is intended to include both solid and liquid formulations of the active compound and one skilled in the art will appreciate that an active ingredient can exist in different preparations depending on the target organ or tissue and on the desired dose and pharmacokinetic parameters.

The term “excipient” as used herein refers to a compound that is useful in preparing a pharmaceutical composition, generally safe, non-toxic and neither biologically nor otherwise undesirable, and includes excipients that are acceptable for veterinary use as well as human pharmaceutical use. The term “excipient” as used herein includes both one and more than one such excipient.

The phrase “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. N-acylsulfonamides have an acidic proton which can be abstracted to form a salt with an organic or inorganic cation.

The preferred pharmaceutically acceptable salts are the salts formed from acetic acid, hydrochloric acid, sulphuric acid, methanesulfonic acid, maleic acid, phosphoric acid, tartaric acid, citric acid, sodium, potassium, calcium, zinc, and magnesium. It should be understood that all references to pharmaceutically acceptable salts include solvent addition forms (solvates) or crystal forms (polymorphs) as defined herein, of the same acid addition salt.

Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs, aqueous solutions, and aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well known suspending agents.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the present invention may be formulated for vaginal administration. Pessaries, tampons, creams, gels, pastes, foams or sprays containing in addition to the active ingredient such carriers as are known in the art to be appropriate.

The compounds of the present invention may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the present invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to a skin-adhesive solid support. The compound of interest can also be combined with a penetration enhancer, e.g., Azone (1-dodecylaza-cycloheptan-2-one). Sustained release delivery systems are inserted subcutaneously into to the subdermal layer by surgery or injection. The subdermal implants encapsulate the compound in a lipid soluble membrane, e.g., silicone rubber, or a biodegradable polymer, e.g., polyactic acid.

Suitable formulations along with pharmaceutical carriers, diluents and excipients are described in Remington: The Science and Practice of Pharmacy 1995, edited by E. W. Martin, Mack Publishing Company, 19th edition, Easton, Pa. A skilled formulation scientist may modify the formulations within the teachings of the specification to provide numerous formulations for a particular route of administration without rendering the compositions of the present invention unstable or compromising their therapeutic activity.

The modification of the present compounds to render them more soluble in water or other vehicle, for example, may be easily accomplished by minor modifications (salt formulation, esterification, etc.), which are well within the ordinary skill in the art. It is also well within the ordinary skill of the art to modify the route of administration and dosage regimen of a particular compound in order to manage the pharmacokinetics of the present compounds for maximum beneficial effect in patients.

The term “therapeutically effective amount” as used herein means an amount required to reduce symptoms of the disease in an individual. The status of an HIV infection can be monitored by measuring viral load (RNA) or monitoring T-cell levels. The dose will be adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.01 and about 100 mg/kg body weight per day should be appropriate in monotherapy and/or in combination therapy. A preferred daily dosage is between about 0.1 and about 500 mg/kg body weight, more preferred 0.1 and about 100 mg/kg body weight and most preferred 1.0 and about 10 mg/kg body weight per day. Thus, for administration to a 70 kg person, the dosage range would be about 7 mg to 0.7 g per day. The daily dosage can be administered as a single dosage or in divided dosages, typically between 1 and 5 dosages per day. Generally, treatment is initiated with smaller dosages which are less than the optimum dose of the compound. Thereafter, the dosage is increased by small increments until the optimum effect for the individual patient is reached. One of ordinary skill in treating diseases described herein will be able, without undue experimentation and in reliance on personal knowledge, experience and the disclosures of this application, to ascertain a therapeutically effective amount of the compounds of the present invention for a given disease and patient.

In embodiments of the invention, the active compound or a salt can be administered in combination with another antiviral agent, such as a nucleoside reverse transcriptase inhibitor, another non-nucleoside reverse transcriptase inhibitor or HIV protease inhibitor. When the active compound or its derivative or salt are administered in combination with another antiviral agent the activity may be increased over the parent compound. When the treatment is combination therapy, such administration may be concurrent or sequential with respect to that of the nucleoside derivatives. “Concurrent administration” as used herein thus includes administration of the agents at the same time or at different times. Administration of two or more agents at the same time can be achieved by a single formulation containing two or more active ingredients or by substantially simultaneous administration of two or more dosage forms with a single active agent.

It will be understood that references herein to treatment extend to prophylaxis as well as to the treatment of existing conditions, and that the treatment of animals includes the treatment of humans as well as other animals. Furthermore, treatment of a HIV infection, as used herein, also includes treatment or prophylaxis of a disease or a condition associated with or mediated by HIV infection, or the clinical symptoms thereof.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

These examples and preparations which follow are provided to enable those skilled in the art to more clearly understand and to practice the present invention. They should not be considered as limiting the scope of the invention, but merely as being illustrative and representative thereof.

### EXAMPLE 1

### 2-[4-Chloro-3-(3-chloro-5-cyano-phenoxy)-2-fluoro-phenoxy]-N-(2-chloro-4-sulfamoyl-phenyl)-acetamide (I-1)

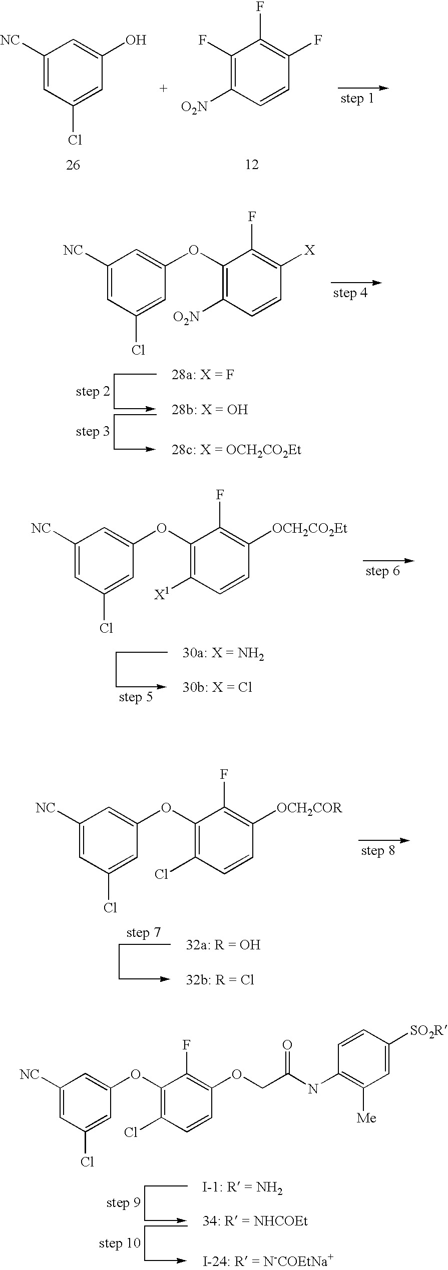

step 1—Solid KOtBu (9.7 g, 1.05 equiv) was added to a solution of 26 (12.7 g, 83 mmol) in THF (350 mL) at 0° C. The mixture was stirred for 20 min and 2,3,4-trifluoronitrobenzene (12, 10 mL, 1.05 equiv) was added. The solution was warmed to RT and aged for 2 h. The mixture was poured into an aqueous ammonium chloride solution and extracted with EtOAc. The organic layer was dried (MgSO₄), and the volatile materials were evaporated. Recrystallization of the resulting solid from MeOH afforded 28a.

step 2—To dry DMSO (125 mL) was added NaH (3.6 g of a 55% suspension, 2.1 equiv) and the resulting suspension was heated to 70° C. for 30 min. The solution was briefly removed from heating bath, and the benzaldoxime (9.5 g, 2 equiv) was added dropwise. The mixture was stirred at 70° C. for an additional 30 min. The thick yellow solution was cooled to RT, and a solution of 28a (12.2 g, 39 mmol) and DMSO (100 mL) was added dropwise. The mixture was heated until the reaction solution became homogenous. The reaction mixture was stirred at RT for 2 h then poured into water. The resulting mixture was extracted with Et₂O, dried and evaporated to afford 28b as a solid that could be recrystallized from MeOH (8.5 g, 70%).

step 3—To a solution of the ethyl bromoacetate (4.85 g, 1.5 equiv) and 28b (6.0 g, 19.4 mmol) in acetone (60 mL) was added anhydrous K₂CO₃ (5.3 g, 2 equiv) and the resulting solution was heated to 60° C. for 2 h. Most of the acetone was removed by evaporation, and the remaining material was partitioned between EtOAc and water. The organic phase was dried (MgSO₄) and the volatile materials were evaporated to afford a solid which was triturated with 10% Et₂O/hexanes to afford 7.2 g (95%) of 28c.

step 4—A mixture of 28c (2.28 g, 5.79 mmol), vanadyl acetylacetonate (0.184 g, 0.12 equiv.) and 5% Pd/C (0.525 g, 0.23 wt/equiv.) in THF (23 mL) was stirred under a H₂ atmosphere maintained with a balloon. The suspension was stirred for 36 h and filtered through CELITE®. The solvents were evaporated and the crude product purified by SiO₂ chromatography eluting with 30% EtOAc/hexanes to afford 1.65 g (78%) of 30a.

step 5-tert-Butyl nitrite (0.674 mL, 1.3 equiv.) and a solution of 30a (1.60 g, 4.38 mmol) and MeCN (8 mL) were added sequentially to a solution of LiCl (0.371 g, 2 equiv.) and CuCl₂ (0.765 g, 1.3 equiv.) in MeCN (22 mL) heated to 60° C. The reaction mixture was maintained at 60° C. for 2 h then quenched with 1 N HCl. The aqueous layer was extracted with EtOAc, and the combined organic extracts were dried (MgSO₄), filtered and evaporated. The crude product was purified by SiO₂ chromatography eluting with 17% EtOAc/hexanes to afford 1.06 g (63%) of 30b.

step 6—A solution of LiOH.H₂O (0.378 g, 1.5 equiv.) and H₂O (23 mL) was added dropwise to an ice-cold solution of 30b (2.31 g, 6.01 mmol) and THF (39 mL). After 30 min., 1 N aqueous HCl was added dropwise to the reaction mixture and the aqueous layer was extracted with EtOAc. The combined organic extracts were dried (MgSO₄), filtered and concentrated in vacuo to afford 1.96 g (91%) of 32a.

step 7—Oxalyl chloride (0.47 mL, 2 equiv.) was added to a solution of 32a (0.96 g, 2.7 mmol) in DCM (8 mL), followed by DMF (2 drops). After 1 h the solvent was removed and the resulting crude acid chloride 32b was used in the next step without further purification.

step 8—To a solution of acid chloride 32b (1.01 g, 2.71 mmol) in acetone (1.3 mL) was added 2-chloro-4-sulfamoylaniline (1.12 g, 2 equiv.). After 1 h the reaction mixture was diluted with H₂O and the resulting solid was filtered, washed with acetone and dried to afford 1.27 g (86%) of I-1.

step 9—To a solution of I-1 (0.729 g, 1.34 mmol), DMAP (0.041 g, 0.25 equiv.) and DMF (1 mL) heated to 90° C. was added propionic anhydride (0.172 mL, 1 equiv.) and the reaction mixture was maintained at 90° C. After 2 h, H₂O (4 mL) and i-PrOH (11 1 mL) were added and the reaction mixture was aged at 60° C. for 1 h. then cooled and the resultant solid was collected after adding H₂O (7 mL). The solid was washed with i-PrOH and H₂O then dried to afford 0.713 g (89%) of 34.

step 10—A solution of 34 (0.666 g, 1.11 mmol) and sodium 2-ethyl hexanoate (0.368 g, 2 equiv.) in THF (4 mL) was heated to 90° C. As THF distilled, butyl acetate was added to replace evaporated THF. The temperature was increased to 127° C. resulting in the formation of a white solid mass. The reaction mixture was cooled to RT and the white solid was filtered and recrystallized from THF to afford 0.280 g (41%) of I-24.

I-2 was prepared by an analogous route except steps 9 and 10 are omitted and in step 8, added 2-chloro-4-sulfamoylaniline was replaced with added 2-methyl-4-sulfamoylaniline.

I-3 was prepared by an analogous route except steps 9 and 10 are omitted and in step 8, added 2-chloro-4-sulfamoylaniline was replaced with 2-bromo-4-sulfamoylaniline.

I-18 and I-19 were prepared analogously using the appropriate aniline derivative in step 8 except in step 1,3-hydroxy-5-chloro-benzonitrile was replaced with 3-hydroxy-5-bromo-benzonitrile.

I-11 and I-12 were prepared analogously using the appropriate aniline derivative in step 8 except in step 1,3-hydroxy-5-chloro-benzonitrile was replaced with 3-hydroxy-5-methoxy-benzonitrile.

### EXAMPLE 2

### 2-[4-Bromo-3-(3-chloro-5-cyano-phenoxy)-2-fluoro-phenoxy]-N-(2-methyl-4-sulfamoyl-phenyl)-acetamide (I-4)

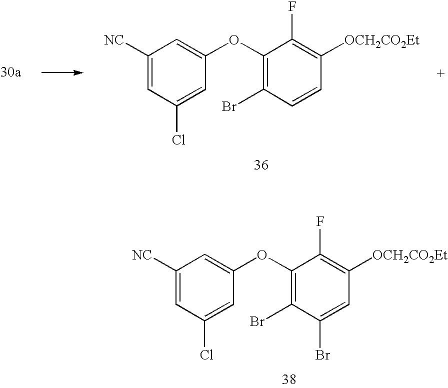

The bromo derivatives were prepared from 30a (1.15 g, 3.16 mmol), LiBr (0.824 g, 3 equiv.), CuBr₂ (0.707 g, 1 equiv.), tert-butyl nitrite (0.450 mL, 1.2 equiv.) and CH₃CN (21 mL) by the procedure described in step 5 of example 1 which afforded a mixture of mono- and dibromo compounds which were separated by SiO₂ chromatography to afford 0.663 g (49%) of 36 and 0.335 g (22%) of 38.

The mono-bromo ester 36a were carried on independently as described in steps 6-8 of example 1 except in step 8,2-chloro-4-sulfamoylaniline (1.12 g, 2 equiv.) was replaced with 2-methyl-4-sulfamoylaniline to afford I-4.

I-5 was prepared from 36 by an analogous route to that used to prepare I-4 except in step 8, 2-methyl-4-sulfamoylaniline was replaced with added 2-chloro-4-sulfamoylaniline

I-17 and I-18 were prepared analogously from [3-(3-cyano-5-methoxy-phenoxy)-2-fluoro-4-nitro-phenoxy]-acetic acid ethyl ester which was prepared as described in examples 1 and 2 except in step 1 of example 1, 3-chloro-5-hydroxy-benzonitrile was replaced with 3-hydroxy-5-methoxy-benzonitrile.

### EXAMPLE 3

### 4-{2-[4-Bromo-3-(3-chloro-5-cyano-phenoxy)-2-fluoro-phenoxy]-acetylamino}-3-methyl-benzamide (I-28)

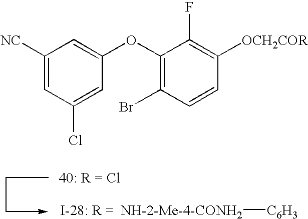

I-28 was prepared from 40 as described in step 8 of example 1 except 2-chloro-4-sulfamoyl-aniline was replaced with 4-carboxamido-2-methyl aniline.

3-Chloro-4-{2-[4-chloro-3-(3-chloro-5-cyano-phenoxy)-2-fluoro-phenoxy]-acetylamino}-benzoic acid (I-27) was prepared analogously by condensation of tert-butyl 4-amino-3-chlorobenzoate with 40 and hydrolysis of the resulting ester under standard conditions.

### EXAMPLE 4

### 2-[3-(3-Chloro-5-cyano-phenoxy)-4-ethyl-2-fluoro-phenoxy]-N-(2-methyl-4-sulfamoyl-phenyl)-acetamide (I-6)

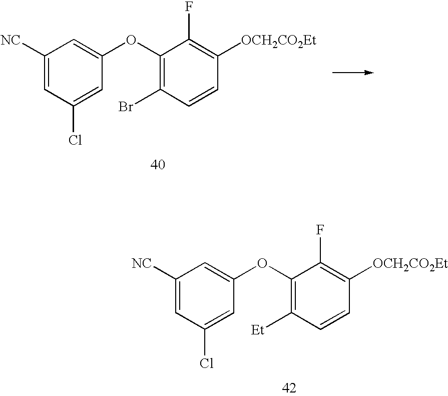

To a solution of 40 (0.663 g, 1.55 mmol) in THF (5 mL) was added sequentially (dppf)PdCl₂CH₂Cl₂ (0.127 g, 0.10 equiv.), ZnEt₂ (2.8 mL, 2.00 equiv. 1.1 M toluene) and dimethyl aminoethanol (0.031 mL, 0.20 eq.). The reaction mixture was initially heated to 65° C., and then cooled to 50° C. for 3 h. The reaction mixture was cooled to RT and quenched with ice-cold sat. aq. NH₄Cl. The aqueous layer was extracted with EtOAc, and the organic extracts were washed with brine and dried (MgSO₄) and evaporated. The crude product was purified by SiO₂ chromatography eluting with 15% EtOAc/hexanes to afford 0.395 g (67%) of 42.

I-6 and I-8 were prepared from 42 as described in steps 6-8 of example 1 using 2-methyl-4-sulfamoylaniline and 2-chloro-4-sulfamoylaniline respectively in the amidation step (step 8).

### EXAMPLE 5

### 2-[3-(3-Chloro-5-cyano-phenoxy)-4-cyclopropyl-2-fluoro-phenoxy]-N-(2-chloro-4-sulfamoyl-phenyl)-acetamide (I-9)

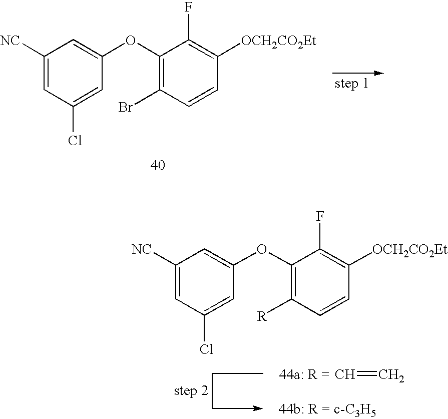

step 1—Tributylvinyltin (0.749 mL, 1.1 equiv.) was added to a solution of 40 (1.00 g, 2.33 mmol), Pd(PPh₃)₄ (0.269 g, 0.1 equiv.) and toluene (10 mL). The reaction mixture was refluxed for 5 h then cooled to RT, filtered through CELITE®. The eluent was partitioned between saturated NH₄Cl (aq) and EtOAc and the organic phase was dried (MgSO₄), filtered and evaporated. The crude product was purified by SiO₂ chromatography eluting with an EtOAc/hexane gradient (17-20% EtOAc) to afford 0.645 g (73%) of 44a.

step 2—N-nitroso-N-methyl urea (1.75 g, 10 equiv.) was added in small portions to an ice-cold mixture of Et₂O (27 mL) and H₂O (15 mL) containing KOH (4.45 g). The resultant yellow mixture was stirred for 1 h at 0° C. The Et₂O phase was decanted into an Erlenmeyer flask containing enough KOH to cover the bottom of the flask then the solution was added to a DCM (15 mL) solution of 44a (0.638 g, 1.69 mmol) and Pd(OAc)₂ (19 mg, 0.05 equiv.). The reaction mixture was stirred at 0° C. for 2 h., filtered through CELITE® and concentrated to afford 0.531 g (80%) of 44b.

I-9 and I-10 were prepared from 44b as described in steps 6-8 of example 1 using 2-chloro-4-sulfamoylaniline and 2-methyl-4-sulfamoylaniline respectively in the amidation step (step 8).

### EXAMPLE 6

### 2-[4-Chloro-3-(3-cyano-5-ethyl-phenoxy)-2-fluoro-phenoxy]-N-(2-chloro-4-sulfamoyl-phenyl)-acetamide (I-20)

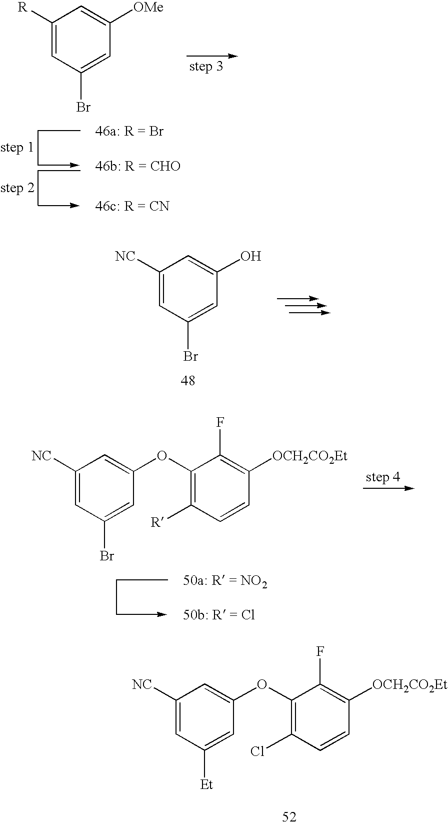

step 1—n-BuLi (2.6 mL of a 1.6 M solution, 1.1 equiv) was added slowly to a solution of 46a (1.0 g, 3.8 mmol) in Et₂O (20 mL) cooled to −78° C. under an N₂ atmosphere. The solution was stirred for 45 min and DMF was added via syringe. The solution was warmed slowly to RT, added to saturated NH₄Cl, and extracted with ether. The organic phase was washed with brine and dried (MgSO₄), filtered and evaporated to afford 0.80 g (98%) of 46b.

step 2—A solution of the aldehyde 46b (12.0 g, 56 mmol), hydroxylamine hydrochloride (19.4 g, 5 equiv), EtOH (100 mL) and pyridine (10 mL) was heated to 65° C. for 16 h. The mixture was cooled to RT, and partitioned between 50% EtOAc/hexanes and water. The organic layer was washed with brine and dried (MgSO₄), filtered and the volatile materials were evaporated to afford 12.4 g (97%) of the oxime. This material was dissolved in anhydrous dioxane (100 mL) and pyridine (26 mL, 6 equiv). The solution was cooled to 0° C., TFAA (15 mL, 2 equiv) was added, and the mixture was allowed to warm to RT. The solution was stirred for 2 d, and warmed to 60° C. for 1 h. The mixture was cooled to RT, and added carefully to ice water. The mixture was extracted with DCM, and the combined organic layers were washed with water, 1 M HCl, and brine. The organic layer was dried (MgSO₄) and evaporated to afford 10.4 g (90%) of 46c,

step 3—Anhydrous collidine (100 mL) was added to a dry flask containing 46c (10.4 g, 49 mmol) and LiI (19.6 g, 3 equiv). The solution was heated under nitrogen to 150° C. overnight, cooled to RT, and poured into an ice cold 1 M HCl solution. The mixture was extracted with a 1:1 EtOAc/hexanes solution, washed with water, and dried (MgSO₄). Concentration in vacuo afforded 8.7 g (89%) of 48.

3-Bromo-5-hydroxy-benzonitrile (48) was converted to 50b by the procedures described in steps I-5 of example 1. Conversion of 50b to 52 (step 4) was carried out by the procedure described in example 4. Final transformation of 52 to I-20 by hydrolysis of the ester, formation of the acid chloride and condensation with an aryl amine was carried out by the standard procedure described in steps 6-8 of example 1.

### EXAMPLE 7

### 2-[4-Chloro-3-(3,5-dicyano-phenoxy)-2-fluoro-phenoxy]-N-(2-methyl-4-sulfamoyl-phenyl)-acetamide (I-14)

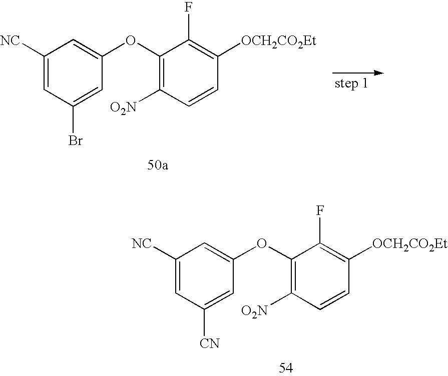

A solution of 50a (2.89 g, 6.59 mmol), Zn(CN)₂ (0.464 g, 0.6 eq.), Pd(PPh₃)₄ (0.761 g, 0.1 eq.) and DMF (33 mL) was heated to 90° C. for 16 h. the reaction mixture was cooled and quenched with 1 N NH₄OH (aq), EtOAc was added and the organic phase was separated, washed with H₂O and dried (MgSO₄), filtered and concentrated in vacuo. The crude product was purified by SiO₂ chromatography eluting with 30% EtOAc/hexanes to afford 0.535 g (22%) of 54.

The aryl bromide 50a was prepared as described in example 6. The bis-cyano ester 54 was converted to I-13 and I-14 as described in steps 6-8 of example 1 using 2-methyl-4-sulfamoylaniline and 2-chloro-4-sulfamoylaniline respectively in the amidation step (step 8).

I-21 and I-22 were prepared from 54 using the Sandmeyer (bromination) reaction as described in example 2 following by steps 6-8 of example 1 using 2-chloro-4-sulfamoyl-aniline and 2-methyl-4-sulfamoyl-aniline in step 8.

### EXAMPLE 8

### 2-[4-Chloro-3-(3-chloro-5-cyano-phenoxy)-phenoxy]-N-(2-methyl-4-sulfamoyl-phenyl)-acetamide (I-15)

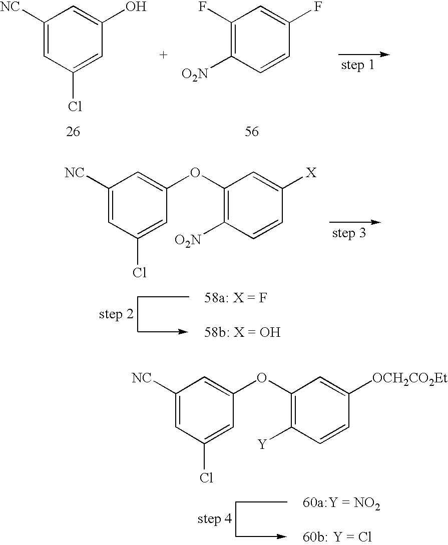

step 1—To a solution of 26 (5.00 g, 32.6 mmol) in THF (34 mL) cooled to 0° C. was added dropwise KO^{t}Bu (36 mL, 1.1 eq., 1.00M solution in THF) after which the solution was warmed to RT. After 1 h, the THF solution was re-cooled to 0° C. and a solution of 2,4-difluoronitrobenzene (56, 5.70 g, 35.8 mmol) in THF (34 mL) was added and the reaction was heated to 50° C. for 3 h. The reaction mixture was cooled to RT and poured into ice-cold H₂O and extracted with EtOAc. The combined extracts were washed with H₂O, dried (MgSO₄), filtered and concentrated in vacuo. The resulting solid was triturated with DCM to afford 6.65 g (70%) of 58a.

step 2—A mixture of 58a (6.65 g, 22.7 mmol), benzaldehyde oxime (4.95 mL, 2 eq.), NaH (1.9 g, 2.1 eq.) and DMSO (136 mL) was converted to 6.60 g (100%) of 58b by utilizing the procedure described in step 2 of example 1

step 3—The phenol 58b (6.60 g, 22.7 mmol) was alkylated with ethyl bromoacetate (3.77 mL, 1.5 eq.), K₂CO₃ (6.27 g, 2.0 eq.) and acetone (91 mL) to afford 7.94 (93%) of 60a utilizing the procedure described in step 3 of example 1.

Replacement of the nitro group with chloride (step 4) and conversion of the ester 60b to anilides I-10 and I-15 with the appropriate aniline was carried out by the procedures in steps 5-8 of example 1.

### EXAMPLE 9

### 2-[4-Chloro-3-(3-cyano-5-difluoromethyl-phenoxy)-2-fluoro-phenoxy]-N-(2-chloro-4-sulfamoyl-phenyl)-acetamide (I-29)

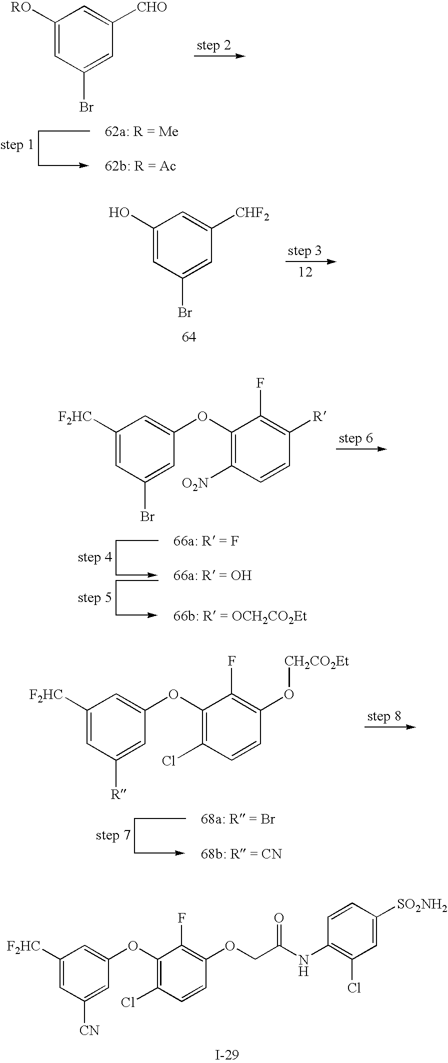

step 1—A solution of BBr₃ (29.1 mL of a 1.0 M solution in DCM, 29.1 mmol) was added slowly to a solution of 62a (2.5 g, 11.62 mmol) in anhydrous DCM (25 mL) maintained under N₂ at −78° C. The orange solution was warmed to RT, stirred for 2 h, and poured onto ice. The mixture was extracted with CH₂Cl₂ (100 mL), and the organic layer was washed with H₂O (50 mL) and brine (50 μL). The solvents were evaporated, and the remaining oil was purified by flash chromatography on silica gel eluting with a EtOAc/hexanes gradient (0% to 20% EtOAc) to provide the desired phenol. To a solution of this phenol in pyridine (10 mL) under argon was slowly added acetic anhydride (0.6 mL, 6.33 mmol). After 2 h, the volatile materials were removed to provide 3-bromo-5-formyl-phenyl acetate (62b, 1.02 g, 40%).

step 2—DAST (1.02 mL, 7.69 mmol) was added to a solution of the 3-bromo-5-formyl-phenyl acetate (62b, 1.1 g, 4.52 mmol) in DCM (5 mL) under nitrogen contained in a NALGENE® bottle. EtOH (0.013 mL, 0.23 mmol) was added, and the mixture was stirred for 16 h. The reaction mixture was then added slowly to an aqueous solution of saturated NaHCO₃. After the bubbling was finished, DCM (50 mL) was added and the layers were separated. The organic layer was washed with brine (30 mL) and dried with anhydrous MgSO₄. The solvent was removed to provide a yellow oil that was placed in a mixture of THF (15 mL) and H₂O (4 mL). LiOH monohydrate (474 mg, 11.3 mmol) was added, and the reaction mixture was stirred at RT for 2 h. The solution was then added dropwise to 5% aqueous HCl (50 mL), and the mixture was extracted with EtOAc (3×30 mL). The combined organic fractions were washed with brine (30 mL), and dried with anhydrous MgSO₄. Evaporation of the volatile materials gave an oil that was purified by flash chromatography on silica gel (0% to 25% EtOAc/hexanes) to provide 800 mg (79%) of 3-bromo-5-difluoromethyl-phenol (64).

The condensation of 3-bromo-5-difluoromethylphenol (64) and 12 (step 3) can be carried out as described in step 1 of example 1. Displacement of the fluoro by hydroxyl (step 4) can be achieved as described in step 2 of example 1. Alkylation of the phenol with ethyl bromoacetate (step 5) can be carried out as described in step 3 of example 1. Conversion of the nitro substituent to a chloride (step 6) can be carried out as described in steps 4 and 5 of example 1. Displacement of the aryl bromide by cyamide (step 7) can be carried out by the procedure described in example 7. Hydrolysis of the ester, conversion to the acid chloride and condensation with 2-methyl-4-sulfamoylaniline (step 8) can be carried out as described in steps 6-8 of example 1 which will afford I-29.

### EXAMPLE 10

### 2-[4-Bromo-3-(3-chloro-5-cyano-phenoxy)-2-fluoro-phenoxy]-N-[2-chloro-4-(2,4-dioxo-imidazolidin-1-yl)-phenyl]-acetamide (I-30)

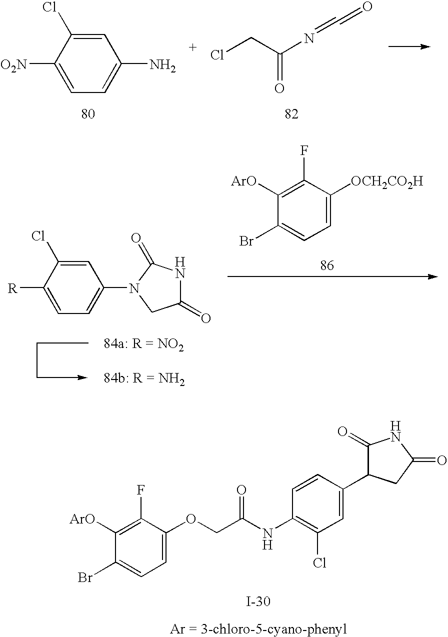

step 1—To a solution of 80 (CASRN 825-41-2, 1.03 g, 5.97 mmol) and dry dioxane at RT was added 2-chloroacetyl isocyanate (82, CASRN 4461-30-7, 0.51 mL, 5.99 mmol) and the resulting solution was stirred at RT for 3 h. A tan solid precipitated after ca. 1 h. DBU (1.78 mL) was added to the mixture and the suspension stirred overnight at RT. An additional aliquot of DBU (1 mL) was added and the solution was stirred for an additional 24 h. The volatile material was evaporated and the dark brown residue dissolved in DCM (100 mL), washed with 1N HCl, dried (MgSO₄), filtered and evaporated to afford 1.13 g of an orange solid which was sonicated in a minimal amount of DCM, filtered and was with DCM to afford 0.5 g of 84a as an orange powder.

step 2—A mixture of 84a (0.5 g, 1095 mmol), Fe powder (0.54 g 9.75 mmol, electrolytic grade, less than 100 mesh), NH₄Cl (4.58 g, 85.7 mmol) and EtOH/H₂O (1:1) was rapidly stirred and heated at 85° C. After 1 h the resulting suspension was filtered through a CELITE® pad and the pad was washed with boiling EtOH. The EtOH solution was cooled to RT and H₂O added portionwise until no further precipitate formed. The solid was filtered, washed with H₂O, filtered and air-dried to afford 0.29 g of 84b as a tan solid. An additional 100 mg was obtained by extraction of the filtrated with EtOAc.

step 3—A solution of 86 (0.1 g, 0.26 mmol, prepared from 36 using the procedure in step 6 of example 1), 84b (0.065 g, 0.28 mmol), EDCI (60 mg, 0.312 mmol) and anhydrous DMF (2 mL) was stirred at RT overnight under inert atmosphere. The resulting solution was diluted with H₂O and twice extracted with EtOAc. The combined extracts were washed with H₂O, dried (MgSO₄), filtered and evaporated. The crude product was heated and sonicated with MeOH and filtered to afford 0.020 g of I-30.

### EXAMPLE 11

### N-[4-(3-Amino-3-methyl-but-1-ynyl)-2-chloro-phenyl]-2-[4-chloro-3-(3-chloro-5-cyano-phenoxy)-2-fluoro-phenoxy]-acetamide (I-32)

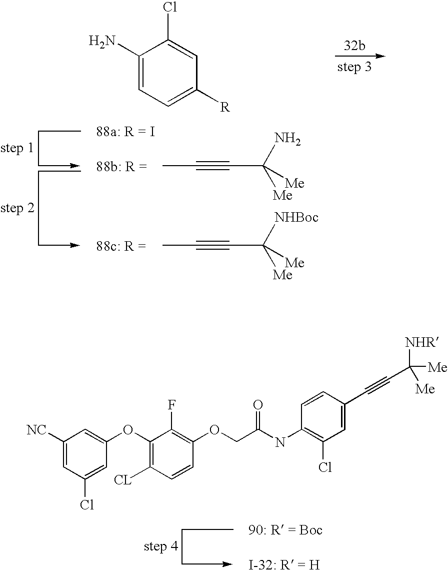

step 1—A round-bottom flask was charged with 88a (CASRN 42016-93-3, 10.7 g, 54.1 mmol), CuI (0.8 g, 0.1 equiv), diethylamine (10.9 mL, 2.5 equiv), 1,1-dimethyl-prop-2-ynylamine (CASRN 2978-58-7, 3.5 g, 1.0 equiv) and 150 mL of THF and argon was bubbled through the resulting mixture for 20 min at RT. To the mixture was added Pd(PPh₃)₄ (4.9 g, 0.1 equiv) and the flask was flushed with nitrogen and heated to 70° C. for 6 h. The volatile solvents were removed in vacuo and the residue purified by SiO₂ chromatography eluting with a MeOH/DCM gradient (0-15% MeOH) to afford 7.0 g of 88b.

step 2—A solution of 88b (0.23 g, 1.08 mmol), di-tert-butyl dicarbonate (0.26 g, 1.1 equiv), TEA (0.224 mL, 1.5 equiv) in THF (5 mL) was stirred for 16 h. The solvent was evaporated in vacuo and the residue purified by SiO₂ chromatography eluting with an EtOAc/hexane gradient (5-30% EtOAc) to afford 0.18 g (54%) of 88c.

step 3—A solution of R-23a (0.22 g, 0.583 mmol), oxalyl chloride (0.1 mL, 2 equiv) DMF (2 drops) in DCM (5 mL) were stirred for 1 h at RT after which the solvents were removed in vacuo.

A solution of 32b and DCM is added to an ice-cold solution of 88c (0.18 g 1 equiv.) and pyridine (1.5 mL) in DCM (2 mL) and the resulting mixture is stirred for 16 h. The reaction mixture is diluted with DCM and is poured into 5% HCl. The organic phase is washed with water and brine, dried (Na₂SO₄), filtered and evaporated. The residue is purified by SiO₂ chromatography eluting with an EtOAc/hexane gradient (5-30% EtOAc) to afford 90.

step 4—To a solution of 90a (3 g, 4.44 mmol) in dioxane is added 4M HCl in dioxane (11 mL, 10 equiv.) and the resulting mixture is stirred for 20 h. The reaction mixture is poured into saturated aqueous NaHCO₃ and the resulting solution extracted with DCM. The combined extracts are washed with water and brine, dried (Na₂SO₄), filtered and the solvents evaporated to afford I-31.

### EXAMPLE 12

### 2-[4-Bromo-3-(3-chloro-5-cyano-phenoxy)-2-fluoro-phenoxy]-N-[2-chloro-4-(3-hydroxy-3-methyl-but-1-ynyl)-phenyl]-acetamide (I-31)

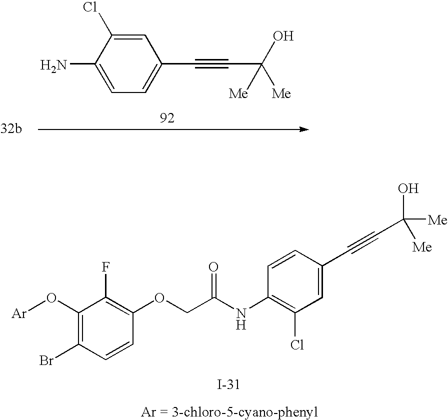

To a solution of 32 (2.6 mmol) and DCM is added a solution of 92 (0.6, 1.1 equiv) and pyridine (5 mL) in DCM (5 mL) and the resulting solution is stirred for 20 h. The reaction mixture is poured into 5% aqueous HCl and is extracted with DCM. The combined extracts are washed with water and brine, dried (Na₂SO₄), filtered and evaporated. The residue is purified by SiO₂ chromatography eluting with an EtOAc/hexane to afford I-31.

### EXAMPLE 13

### HIV-1 Reverse Transcriptase Assay Inhibitor IC₅₀ Determination

RNA-dependent DNA polymerase activity was measured using a biotinylated primer oligonucleotide and tritiated dNTP substrate. Newly synthesized DNA was quantified by capturing the biotinylated primer molecules on streptavidin coated Scintillation Proximity Assay (SPA) beads (Amersham). The sequences of the polymerase assay substrate were: 18nt DNA primer, 5′-Biotin/GTC CCT GTT CGG GCG CCA-3′; 47nt RNA template, 5′-GGG UCU CUC UGG UUA GAC CAC UCU AGC AGU GGC GCC CGA ACA GGG AC-3′. The biotinylated DNA primer was obtained from the Integrated DNA Technologies Inc. and the RNA template was synthesized by Dharmacon. The DNA polymerase assay (final volume 50 μl) contained 32 nM biotinylated DNA primer, 64 nM RNA substrate, dGTP, dCTP, dTTP (each at 5 μM), 103 nM [³H]-dATP (specific activity=29 μCi/mmol), in 45 mM Tris-HCl, pH 8.0, 45 mM NaCl, 2.7 mM Mg(CH₃COO)₂, 0.045% Triton X-100 w/v, 0.9 mM EDTA. The reactions contained 5 μl of serial compound dilutions in 100% DMSO for IC₅₀ determination and the final concentrations of DMSO were 10%. Reactions were initiated by the addition of 30 μl of the HIV-RT enzyme (final concentrations of I-3 nM). Protein concentrations were adjusted to provide linear product formation for at least 30 min of incubation. After incubation at 30° for 30 min, the reaction was quenched by addition of 50 μl of 200 mM EDTA (pH 8.0) and 2 mg/ml SA-PVT SPA beads (Amersham, RPNQ0009, reconstituted in 20 mM Tris-HCl, pH 8.0, 100 mM EDTA and 1% BSA). The beads were left to settle overnight and the SPA signals were counted in a 96-well top counter-NXT (Packard). IC₅₀ values were obtained by sigmoidal regression analysis using GraphPad Prism 3.0 (GraphPad Software, Inc.).

### EXAMPLE 14

Pharmaceutical compositions of the subject compounds for administration via several routes were prepared as described in this Example.

Composition for Oral Administration (A)

| Ingredient | % wt./wt. |
|---|---|
| Active ingredient | 20.0% |
| Lactose | 79.5% |
| Magnesium stearate | 0.5% |

The ingredients are mixed and dispensed into capsules containing about 100 mg each; one capsule would approximate a total daily dosage.

Composition for Oral Administration (B)

| Ingredient | % wt./wt. |
|---|---|
| Active ingredient | 20.0% |
| Magnesium stearate | 0.5% |
| Crosscarmellose sodium | 2.0% |
| Lactose | 76.5% |
| PVP (polyvinylpyrrolidine) | 1.0% |

The ingredients are combined and granulated using a solvent such as methanol. The formulation is then dried and formed into tablets (containing about 20 mg of active compound) with an appropriate tablet machine.

Composition for Oral Administration (C)

| Ingredient | % wt./wt. | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | ml |
| Colorings | 0.5 | mg |
| Distilled water | q.s. to 100 | ml |

The ingredients are mixed to form a suspension for oral administration.

Parenteral Formulation (D)

| Ingredient | % wt./wt. |
|---|---|
| Active ingredient | 0.25 g |
| Sodium Chloride | qs to make isotonic |
| Water for injection to | 100 ml |

The active ingredient is dissolved in a portion of the water for injection. A sufficient quantity of sodium chloride is then added with stirring to make the solution isotonic. The solution is made up to weight with the remainder of the water for injection, filtered through a 0.2 micron membrane filter and packaged under sterile conditions.

Suppository Formulation (E)

| Ingredient | % wt./wt. |
|---|---|
| Active ingredient | 1.0% |
| Polyethylene glycol 1000 | 74.5% |
| Polyethylene glycol 4000 | 24.5% |

The ingredients are melted together and mixed on a steam bath, and poured into molds containing 2.5 g total weight.

The features disclosed in the foregoing description, or the following claims, expressed in their specific forms or in terms of a means for performing the disclosed function, or a method or process for attaining the disclosed result, as appropriate, may, separately, or in any combination of such features, be utilized for realizing the invention in diverse forms thereof.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

We claim:

1. A compound according to formula I 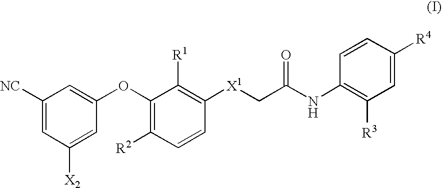
wherein:
R¹ is fluorine or hydrogen;
R² is hydrogen, chloro, bromo, C₁₋₃ alkyl, C₃₋₅ cycloalkyl or C₁₋₃ alkoxy;
X¹ is O or S;
X² is chloro, bromo, cyano, C₁₋₃ alkoxy, C₃₋₅ cycloalkyl or C₁₋₃ haloalkyl;
R³ is selected from the group consisting of C₁₋₆ alkyl, C₁₋₃ haloalkyl, C₁₋₆ alkoxy, C₁₋₆ haloalkoxy, C₃₋₅ cycloalkyl, halogen and cyano; 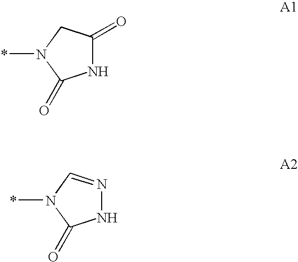
R⁴ is SO₂NHR^{5a}R^{6a}, COX⁴, —C≡CC(Me)₂R⁸, A1 or A2;
X⁴ is OH or NR^{5b}R^{6b};
R^{5a} and R^{6a}
(i) taken independently, one of R^{5a} and R^{6a} is hydrogen or C₁₋₆ alkyl and the other of R^{5a} and R^{6a} is selected from the group consisting of hydrogen, C₁₋₆ alkyl and —C(═O)R⁷;
(ii) taken together with the nitrogen atom to which they are attached form an azetidine, pyrrolidine, piperidine or azepine ring said azetidine, pyrrolidine, piperidine or azepine ring optionally substituted with hydroxy, amino, C₁₋₃ alkylamine or C₁₋₃ dialkylamine; or,
(iii) taken together are (CH₂)₂—X³—(CH₂)₂;
R^{5b} and R^{6b}
(i) are independently selected from the group consisting of hydrogen, C₁₋₆ alkyl, C₁₋₆ hydroxyalkyl, C₁₋₆ carboxyalkyl, (CH₂)ᵣNR^{5c}R^{6c} wherein r is 2 to 6, and SO₂—C₁₋₆ alkyl;
(ii) taken together with the nitrogen atom to which they are attached form an azetidine, pyrrolidine, piperidine or azepine ring said azetidine, pyrrolidine, piperidine or azepine ring optionally substituted hydroxy, amino, C₁₋₃ alkylamine or C₁₋₃ dialkylamine; or,
(iii) taken together are (CH₂)₂X³(CH₂)₂;
R^{5c} and R^{6c}
(i) are independently from the group consisting of hydrogen and C₁₋₆ alkyl,
(ii) together with the nitrogen atom to which they are attached form an azetidine, pyrrolidine, piperidine or azepine ring said azetidine, pyrrolidine, piperidine or azepine ring optionally substituted hydroxy, amino, C₁₋₃ alkylamine or C₁₋₃ dialkylamine, or
(iii) taken together are (CH₂)₂X³(CH₂)₂;
R^{5d} and R^{6d} are independently in each occurrence hydrogen, C₁₋₆ alkyl, C₁₋₆ hydroxyalkyl or C₁₋₆ carboxyalkyl;
X³ is independently in each occurrence O, S(O)ₚ or NR¹⁰;
R⁷ is hydrogen or C₁₋₆ alkyl;
R⁸ is OH, NR^{5d}R^{6d}, CO₂H, CONR^{5d}R^{5d} or C(═O)NR^{9a}C(═NR^{9b})NR^{9c}R^{9d};
R^{9a}, R^{9b}, R^{9c} and R^{9d} are (i) independently hydrogen or C₁₋₆ alkyl or (ii) R^{9a} and R^{9d} are independently hydrogen or C₁₋₆ alkyl and R^{9b} and R^{9c} together are C₂₋₄ alkylene;
R¹⁰ is hydrogen, C₁₋₆ alkyl or C₁₋₆ acyl;
p is 0 to 2; or,
a pharmaceutically acceptable salt thereof.

2. A compound according to claim 1 wherein R⁴ is SONHR^{5a}R^{6a} or COX⁴.

3. A compound according to claim 2 wherein X¹ is O, R¹ is fluoro, R³ is chloro, bromo, or methyl, and R⁴ is SONHR^{5a}R^{6a}.

4. A compound according to claim 3 wherein R² is methyl, ethyl, methoxy, chloro or bromo, and X² is chloro, bromo, difluoromethyl or cyano.

5. A compound according to claim 4 wherein R^{5a} is hydrogen, R^{6a} is hydrogen or R⁷C(═O) and R⁷ is C₁₋₁₀ alkyl.

6. A compound according to claim 2 wherein X¹ is O, R¹ is fluoro, R³ is chloro, bromo or methyl and R⁴ is CONR^{5b}R^{6b}.

7. A compound according to claim 6 wherein R² is methyl, ethyl, methoxy, chloro or bromo and X² is chloro, bromo, difluoromethyl or cyano.

8. A compound according to claim 2 wherein X¹ is S, X² is chloro, bromo, difluoromethyl or cyano, R¹ is fluoro, R² is methyl, ethyl, methoxy, chloro or bromo, and R³ is chloro, bromo or methyl.

9. A compound according to claim 1 wherein X² is difluoromethyl.

10. A compound according to claim 9 wherein R⁴ is SO₂NHR^{5a}R^{6a} or COX⁴.

11. A compound according to claim 10 wherein X¹ is O, R¹ is fluoro, R² is methyl, ethyl, methoxy, chloro or bromo, R³ is chloro, bromo or methyl and R⁴ is SO₂NHR^{5a}R^{6a}.

12. A compound according to claim 1 wherein claim 1 wherein X¹ is O, R¹ is fluoro, R³ is chloro, bromo, or methyl, and R⁴ is —C≡CC(Me)₂R⁸.

13. A compound according to claim 12 wherein R⁸ is CO₂H.

14. A compound according to claim 12 wherein R⁸ is NR^{5c}R^{5d}.

15. A compound according to claim 1 wherein claim 1 wherein X¹ is O, R¹ is fluoro, R³ is chloro, bromo, or methyl, and R⁴ is A1 or A2.

16. A compound according to claim 9 wherein X¹ is O, R¹ is fluoro, R² is methyl, ethyl, methoxy, chloro or bromo, R³ is chloro, bromo or methyl and R⁴ is CONR^{5b}R^{6b}.

17. A compound according to claim 1 selected from the group consisting of:
2-[4-Chloro-3-(3-chloro-5-cyano-phenoxy)-2-fluoro-phenoxy]-N-(2-chloro-4-sulfamoyl-phenyl)-acetamide;
2-[4-Chloro-3-(3-chloro-5-cyano-phenoxy)-2-fluoro-phenoxy]-N-(2-methyl-4-sulfamoyl-phenyl)-acetamide;
N-(2-Bromo-4-sulfamoyl-phenyl)-2-[4-chloro-3-(3-chloro-5-cyano-phenoxy)-2-fluoro-phenoxy]-acetamide;
2-[4-Bromo-3-(3-chloro-5-cyano-phenoxy)-2-fluoro-phenoxy]-N-(2-methyl-4-sulfamoyl-phenyl)-acetamide;
2-[4-Bromo-3-(3-chloro-5-cyano-phenoxy)-2-fluoro-phenoxy]-N-(2-chloro-4-sulfamoyl-phenyl)-acetamide;
2-[3-(3-Chloro-5-cyano-phenoxy)-4-ethyl-2-fluoro-phenoxy]-N-(2-methyl-4-sulfamoyl-phenyl)-acetamide;
2-[3-(3-Chloro-5-cyano-phenoxy)-4-ethyl-2-fluoro-phenoxy]-N-(2-chloro-4-sulfamoyl-phenyl)-acetamide;
2-[3-(3-Chloro-5-cyano-phenoxy)-4-cyclopropyl-2-fluoro-phenoxy]-N-(2-chloro-4-sulfamoyl-phenyl)-acetamide;
2-[3-(3-Chloro-5-cyano-phenoxy)-4-cyclopropyl-2-fluoro-phenoxy]-N-(2-methyl-4-sulfamoyl-phenyl)-acetamide;
2-[4-Chloro-3-(3-chloro-5-cyano-phenoxy)-phenoxy]-N-(2-chloro-4-sulfamoyl-phenyl)-acetamide;
2-[4-Chloro-3-(3-cyano-5-methoxy-phenoxy)-2-fluoro-phenoxy]-N-(2-methyl-4-sulfamoyl-phenyl)-acetamide;
2-[4-Chloro-3-(3-cyano-5-methoxy-phenoxy)-2-fluoro-phenoxy]-N-(2-chloro-4-sulfamoyl-phenyl)-acetamide;
2-[4-Chloro-3-(3,5-dicyano-phenoxy)-2-fluoro-phenoxy]-N-(2-methyl-4-sulfamoyl-phenyl)-acetamide;
2-[4-Chloro-3-(3,5-dicyano-phenoxy)-2-fluoro-phenoxy]-N-(2-chloro-4-sulfamoyl-phenyl)-acetamide;
2-[4-Chloro-3-(3-chloro-5-cyano-phenoxy)-phenoxy]-N-(2-methyl-4-sulfamoyl-phenyl)-acetamide;
2-[4-Bromo-3-(3-cyano-5-methoxy-phenoxy)-2-fluoro-phenoxy]-N-(2-methyl-4-sulfamoyl-phenyl)-acetamide;
2-[4-Bromo-3-(3-cyano-5-methoxy-phenoxy)-2-fluoro-phenoxy]-N-(2-chloro-4-sulfamoyl-phenyl)-acetamide;
2-[3-(3-Bromo-5-cyano-phenoxy)-4-chloro-2-fluoro-phenoxy]-N-(2-methyl-4-sulfamoyl-phenyl)-acetamide;
2-[3-(3-Bromo-5-cyano-phenoxy)-4-chloro-2-fluoro-phenoxy]-N-(2-chloro-4-sulfamoyl-phenyl)-acetamide;
2-[4-Chloro-3-(3-cyano-5-ethyl-phenoxy)-2-fluoro-phenoxy]-N-(2-chloro-4-sulfamoyl-phenyl)-acetamide;
2-[4-Bromo-3-(3,5-dicyano-phenoxy)-2-fluoro-phenoxy]-N-(2-chloro-4-sulfamoyl-phenyl)-acetamide;
2-[4-Bromo-3-(3,5-dicyano-phenoxy)-2-fluoro-phenoxy]-N-(2-methyl-4-sulfamoyl-phenyl)-acetamide;
2-[4-Bromo-3-(3-chloro-5-cyano-phenoxy)-2-fluoro-phenoxy]-N-(2-chloro-4-propionylsulfamoyl-phenyl)-acetamide, sodium salt;
2-[4-Bromo-3-(3-chloro-5-cyano-phenoxy)-2-fluoro-phenoxy]-N-(2-methyl-4-propionylsulfamoyl-phenyl)-acetamide, sodium salt;
2-[4-Chloro-3-(3-cyano-5-cyclopropyl-phenoxy)-2-fluoro-phenoxy]-N-(2-methyl-4-sulfamoyl-phenyl)-acetamide;
2-[4-Chloro-3-(3-cyano-5-cyclopropyl-phenoxy)-2-fluoro-phenoxy]-N-(2-chloro-4-sulfamoyl-phenyl)-acetamide;
3-Chloro-4-{2-[4-chloro-3-(3-chloro-5-cyano-phenoxy)-2-fluoro-phenoxy]-acetylamino}-benzoic acid;
4-{2-[4-Bromo-3-(3-chloro-5-cyano-phenoxy)-2-fluoro-phenoxy]-acetylamino}-3-methyl-benzamide;
2-[4-Chloro-3-(3-cyano-5-difluoromethyl-phenoxy)-2-fluoro-phenoxy]-N-(2-chloro-4-sulfamoyl-phenyl)-acetamide;
2-[4-Bromo-3-(3-chloro-5-cyano-phenoxy)-2-fluoro-phenoxy]-N-[2-chloro-4-(2,4-dioxo-imidazolidin-1-yl)-phenyl]-acetamide;
2-[4-Bromo-3-(3-chloro-5-cyano-phenoxy)-2-fluoro-phenoxy]-N-[2-chloro-4-(3-hydroxy-3-methyl-but-1-ynyl)-phenyl]-acetamide; and,
N-[4-(3-Amino-3-methyl-but-1-ynyl)-2-chloro-phenyl]-2-[4-chloro-3-(3-chloro-5-cyano-phenoxy)-2-fluoro-phenoxy]-acetamide.

18. A method for treating an HIV infection, or preventing an HIV infection, or treating AIDS or ARC, comprising administering to a host in need thereof a therapeutically effective amount of a compound according to claim 1.

19. A method for treating an HIV infection, or preventing an HIV infection, or treating AIDS or ARC, comprising administering to a host in need thereof a therapeutically effective amount of a compound according to claim 2.

20. A method for treating HIV infection according to claim 18 further comprising co-administering at least one compound selected from the group consisting of HIV protease inhibitors, nucleoside reverse transcriptase inhibitors, non-nucleoside reverse transcriptase inhibitors, CCR5 antagonists and viral fusion inhibitors.

21. A method according to claim 20 wherein the reverse transcriptase inhibitor is selected from the group consisting of zidovudine, lamivudine, didanosine, zalcitabine, stavudine, rescriptor, sustiva and viramune, efavirenz, nevirapine or delavirdine and/or the protease inhibitor is selected from the group consisting of saquinavir, ritonavir, nelfinavir, indinavir, amprenavir, and lopinavir and/or the viral fusion inhibitor is enfuvirtide.

22. A method for inhibiting HIV reverse transcriptase in a host infected with HIV comprising administering a compound according to claim 1.

23. A method for inhibiting HIV reverse transcriptase in a host infected with HIV comprising administering a compound according to claim 2.

24. A method according to claim 22 wherein the host is infected with a strain of HIV expressing a reverse transcriptase with at least one mutation compared to wild type HIV.

25. A method according to claim 24 wherein said strain of HIV exhibits reduced susceptibility to efavirenz, nevirapine or delavirdine.

26. A pharmaceutical composition for treating an HIV infection, or preventing an HIV infection, or treating AIDS or ARC, comprising a therapeutically effective quantity of a compound according to claim 1 and at least one carrier, excipient or diluent.

27. A process for preparing a compound of formula I which process comprises the steps of: 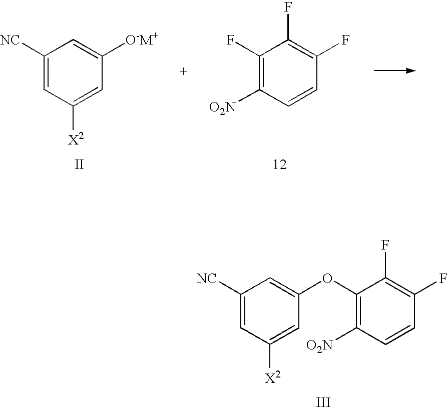
(i) condensing the salt of a substituted 3-cyanophenol II with 2,3,4-trifluoronitrobenzene to afford a biaryl ether III; 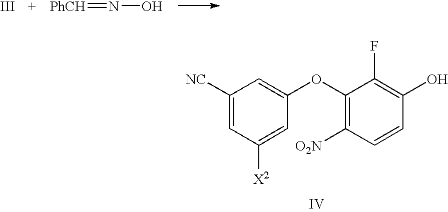
(ii) reacting II with benzaldoxime and base in an inert solvent to afford phenol IV; 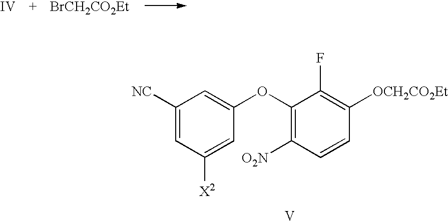
(iii) alkylating the phenol IV with an alkyl bromoacetate salt or an equivalent acetic acid synthetic equivalent to afford V; 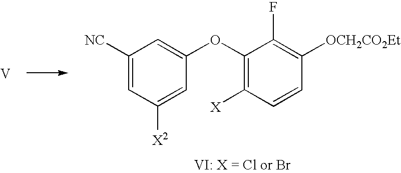
(iv) converting the nitro group in V into a chloride or bromide VI (X=Cl or Br) by the three step sequence of reduction of the nitro to an amine, diazotization of the amine and displacement of the diazo group with chloride or bromide and optionally displacing the bromide thus formed with a dialkylzinc in the presence of a palladium catalyst to afford VI (X=alkyl); 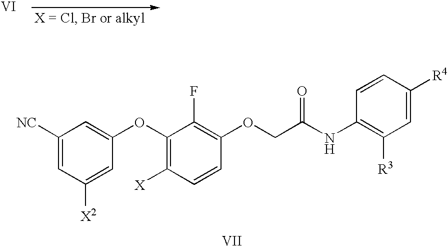
(iv) converting the ester VI into the corresponding 4-sulfamoyl-anilide VII (R⁴=SO₂NHR^{5a}R^{6a}) or 4 carbamoyl-anilide VII (R⁴=CONR^{5b}R^{6b}).
